(19) United States
(12) Patent Application Publication
(10) Pub. No.: US 2014/0242182 A1
(43) Pub. Date: Aug. 28, 2014

(54) FORMULATIONS THAT STABILIZE PROTEINS

(71) Applicants: Sean A. Evans, Acton, MA (US); Greg J. Allard, Milford, MA (US); Nicholas C. Masiello, Uxbridge, MA (US)

(72) Inventors: Sean A. Evans, Acton, MA (US); Greg J. Allard, Milford, MA (US); Nicholas C. Masiello, Uxbridge, MA (US)

(73) Assignee: rEVO Biologics, Inc, Fromingham, MA (US)

(21) Appl. No.: 14/131,289

(22) PCT Filed: Jul. 6, 2012
(86) PCT No.: PCT/US12/45699

§ 371 Date: May 19, 2014

Related U.S. Application Data
(60) Provisional application No. 61/505,354, filed on Jul. 7, 2011.

(51) Int. Cl. A61K 47/02; A61K 38/57

(52) U.S. Cl. CPC A61K 47/02; A61K 38/57
USPC 424/535; 514/14.7; 530/393

(57) ABSTRACT

In one aspect, the disclosure provides formulations that stabilize proteins, wherein the formulations comprise a buffer. In some embodiments, the buffer comprises potassium mono-hydrogen-phosphate and potassium di-hydrogen-phosphate, or the buffer comprises sodium mono-hydrogen-phosphate and sodium di-hydrogen-phosphate. In some embodiments, the protein is a therapeutic protein. In some embodiments, the therapeutic protein is antithrombin.

### FIELD OF THE INVENTION

The disclosure provides formulations that stabilize proteins, including therapeutic proteins such as antithrombin.

### BACKGROUND OF THE INVENTION

The limited stability of therapeutic proteins is a general problem in the pharmaceutical industry both during the production phase and during the storage of the final therapeutic protein formulation that is to be administered. For instance, during the production of therapeutic proteins (e.g., synthetically, recombinantly or transgenically), proteins are often stored for long periods of time between the various purification and processing steps, and formulation components can have an influence on the stability of therapeutic proteins.

### SUMMARY OF THE INVENTION

In one aspect, the disclosure provides formulations that stabilize proteins, such as therapeutic proteins. In some embodiments, the formulation comprises a buffer, wherein the buffer comprises mono-hydrogen-phosphate and di-hydrogen-phosphate, and wherein the mono-hydrogen-phosphate and di-hydrogen-phosphate have the same counter ion. In some embodiments, the counter ion is sodium or potassium. In some embodiments, the formulation comprises a buffer, wherein the buffer comprises potassium mono-hydrogen-phosphate and potassium di-hydrogen-phosphate. In some embodiments, the formulation comprises a buffer, wherein the buffer comprises sodium mono-hydrogen-phosphate and sodium di-hydrogen-phosphate.

In some embodiments, the formulation comprises a buffer, wherein the buffer essentially consists of potassium mono-hydrogen-phosphate and potassium di-hydrogen-phosphate. In some embodiments, the formulation comprises a buffer, wherein the buffer essentially consists of sodium mono-hydrogen-phosphate and sodium di-hydrogen-phosphate. In some embodiments, the formulation does not include both sodium mono-hydrogen-phosphate and potassium di-hydrogen-phosphate. In some embodiments, the formulation does not include both potassium mono-hydrogen-phosphate and sodium di-hydrogen-phosphate.

In some embodiments, the formulations comprise a therapeutic protein. In some embodiments, the therapeutic protein is antithrombin.

In one aspect the disclosure provides a formulation comprising a therapeutic protein and a buffer, wherein the buffer comprises potassium mono-hydrogen-phosphate and potassium di-hydrogen-phosphate, or wherein the buffer comprises sodium mono-hydrogen-phosphate and sodium di-hydrogen-phosphate. In some embodiments of any of the formulations disclosed herein, the buffer has a concentration of between 10 mM and 100 mM. In some embodiments of any of the formulations disclosed herein, the buffer has a concentration of 50 mM. In some embodiments of any of the formulations disclosed herein, the formulation further comprises potassium chloride. In some embodiments of any of the formulations disclosed herein, the potassium chloride has a concentration of between 100 and 150 mM. In some embodiments of any of the formulations disclosed herein, the potassium chloride has a concentration of 120 mM. In some embodiments of any of the formulations disclosed herein, the pH of the formulation is between 7.5 and 8.5. In some embodiments of any of the formulations disclosed herein, the pH of the formulation is 8. In some embodiments of any of the formulations disclosed herein, the therapeutic protein is antithrombin. In some embodiments of any of the formulations disclosed herein, the formulation comprises clarified milk product. In some embodiments of any of the formulations disclosed herein, the formulation includes additional proteins.

In one aspect the disclosure provides formulations comprising antithrombin. In some embodiments of any of the formulations comprising antithrombin disclosed herein, the antithrombin maintains at least 90% of heparin binding functionality after storage at 2-8° C. for three months as compared to heparin binding functionality prior to storage. In some embodiments of any of the formulations comprising antithrombin disclosed herein, the increase in the amount of antithrombin (by weight) that is in an aggregated form after storage at 2-8° C. for three months is less than 3-fold as compared to the amount of antithrombin (by weight) that is in an aggregated form prior to storage. In some embodiments of any of the formulations comprising antithrombin disclosed herein, the increase in the amount of oxidation of antithrombin after storage at 2-8° C. for three months is less than 2-fold as compared to the amount of oxidation of antithrombin prior to storage.

In one aspect the disclosure provides a method for generating a formulation that stabilizes therapeutic protein, the method comprising providing a solution comprising a buffer, wherein the buffer comprises potassium mono-hydrogen-phosphate and potassium di-hydrogen-phosphate, or

wherein the buffer comprises sodium mono-hydrogen-phosphate and sodium di-hydrogen-phosphate, and adding therapeutic protein to the solution resulting in a formulation that stabilizes the therapeutic protein.

In one aspect the disclosure provides a method for generating a formulation that stabilizes therapeutic protein, the method comprising providing a solution comprising therapeutic protein, and adding a buffer to the solution resulting in a formulation that stabilizes therapeutic protein, wherein the buffer comprises potassium mono-hydrogen-phosphate and potassium di-hydrogen-phosphate, or wherein the buffer comprises sodium mono-hydrogen-phosphate and sodium di-hydrogen-phosphate.

In some embodiments of any of the methods disclosed herein, the resulting concentration of the buffer is 50 mM. In some embodiments of any of the methods disclosed herein, the formulation further comprises potassium chloride. In some embodiments of any of the methods disclosed herein, the resulting pH of the solution is a pH of 8. In some embodiments of any of the methods disclosed herein, the therapeutic protein is antithrombin.

In one aspect the disclosure provides a method for generating a formulation that stabilizes antithrombin, the method comprising separating antithrombin from a milk composition comprising antithrombin resulting in a solution comprising antithrombin, pasteurizing the solution comprising antithrombin, exchanging the solution comprising antithrombin for a buffer, wherein the buffer comprises potassium mono-hydrogen-phosphate and potassium di-hydrogen-phosphate, or wherein the buffer comprises sodium mono-hydrogen-phosphate and sodium di-hydrogen-phosphate, thereby generating a formulation that stabilizes antithrombin.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the Figures. The invention is capable of other embodiments and of being practiced or of being carried out in various ways. Also, the phraseology and terminology used herein is for the purpose of description and should not be regarded as limiting.

### BRIEF DESCRIPTION OF THE DRAWINGS

FIG. 1 shows the oxidation status of antithrombin after freeze/thaw in a variety of buffers.

FIG. 2 shows the heparin affinity of antithrombin after freeze/thaw in a variety of buffers.

FIG. 3 shows the aggregation of antithrombin after freeze/thaw in a variety of buffers.

FIG. 4 shows the oxidation status of antithrombin after storage at 2-8° C. in a variety of buffers.

FIG. 5 shows the heparin affinity of antithrombin after storage at 2-8° C. in a variety of buffers.

FIG. 6 shows the aggregation of antithrombin after storage at 2-8° C. in a variety of buffers.

FIG. 7 provides an overview of the stability parameters of antithrombin after freeze/thaw in phosphate systems.

FIG. 8 provides an overview of the stability parameters of antithrombin after storage at 2-8° C. for one month in phosphate systems.

FIG. 9 provides an overview of the stability parameters of antithrombin after storage at 2-8° C. for three months in phosphate systems.

FIG. 10 shows an overview of the stability parameters of antithrombin after storage at 2-8° C. for one month in a variety of buffers.

FIG. 11 shows the oxidation status of antithrombin after freeze/thaw in a variety of buffers that include potassium chloride.

FIG. 12 shows the heparin affinity of antithrombin after freeze/thaw in a variety of buffers that include potassium chloride.

FIG. 13 shows the aggregation of antithrombin after freeze/thaw in a variety of buffers that include potassium chloride.

FIG. 14 provides an overview of the stability parameters of antithrombin after freeze/thaw in a variety of buffers that include potassium chloride.

FIG. 15 shows the oxidation status of antithrombin after storage at 2-8° C. in a variety of buffers that include potassium chloride.

FIG. 16 shows the heparin affinity of antithrombin after storage at 2-8° C. in a variety of buffers that include potassium chloride.

FIG. 17 shows the aggregation of antithrombin after storage at 2-8° C. in a variety of buffers that include potassium chloride.

FIG. 18 provides an overview of the stability parameters of antithrombin after storage at 2-8° C. in a variety of buffers that include potassium chloride.

FIG. 19 shows the oxidation of antithrombin over a period of 24 months.

FIG. 20 shows the aggregation of antithrombin over a period of 24 months.

FIG. 21 shows the heparin affinity of antithrombin over a period of 24 months.

FIG. 22 shows the throughput data of a heparin eluate using the conventional process.

FIG. 23 shows the throughput data of a heparin eluate using the clarified milk.

FIG. 24 shows an SDS page of the heparin eluates.

FIG. 25 shows the stability of antithrombin formulation lot #300-21-DS.

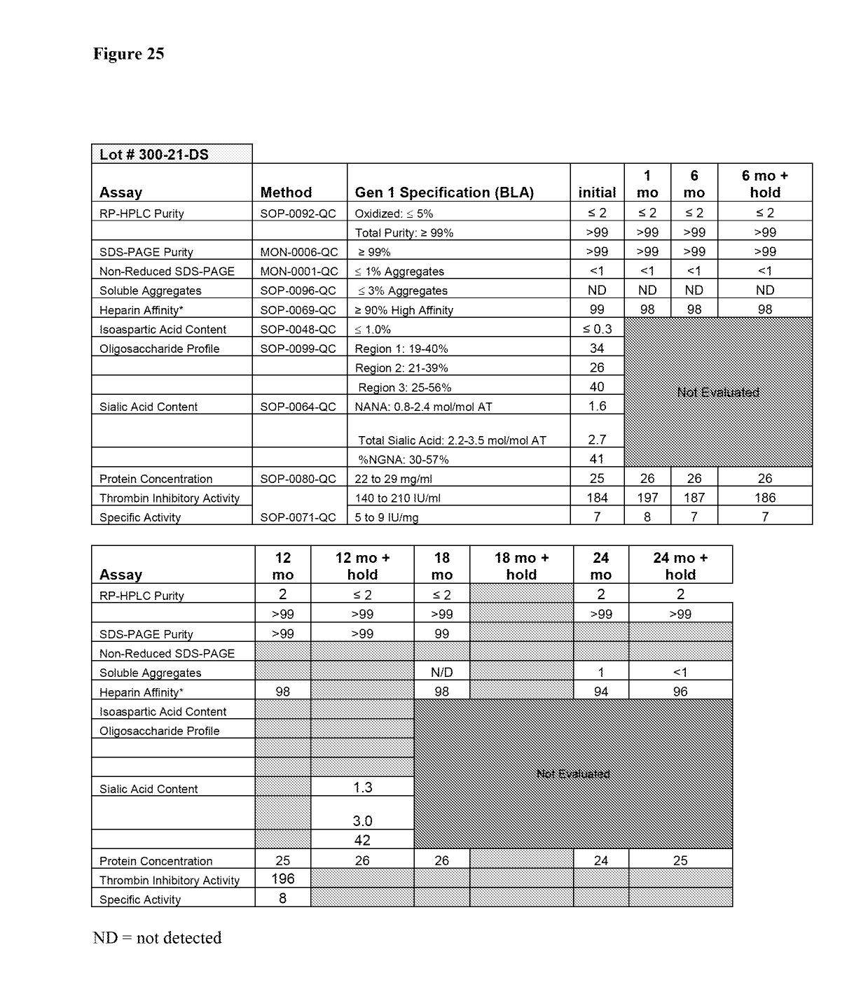

FIG. 26 shows the stability of antithrombin formulation lot #300-22-DS.

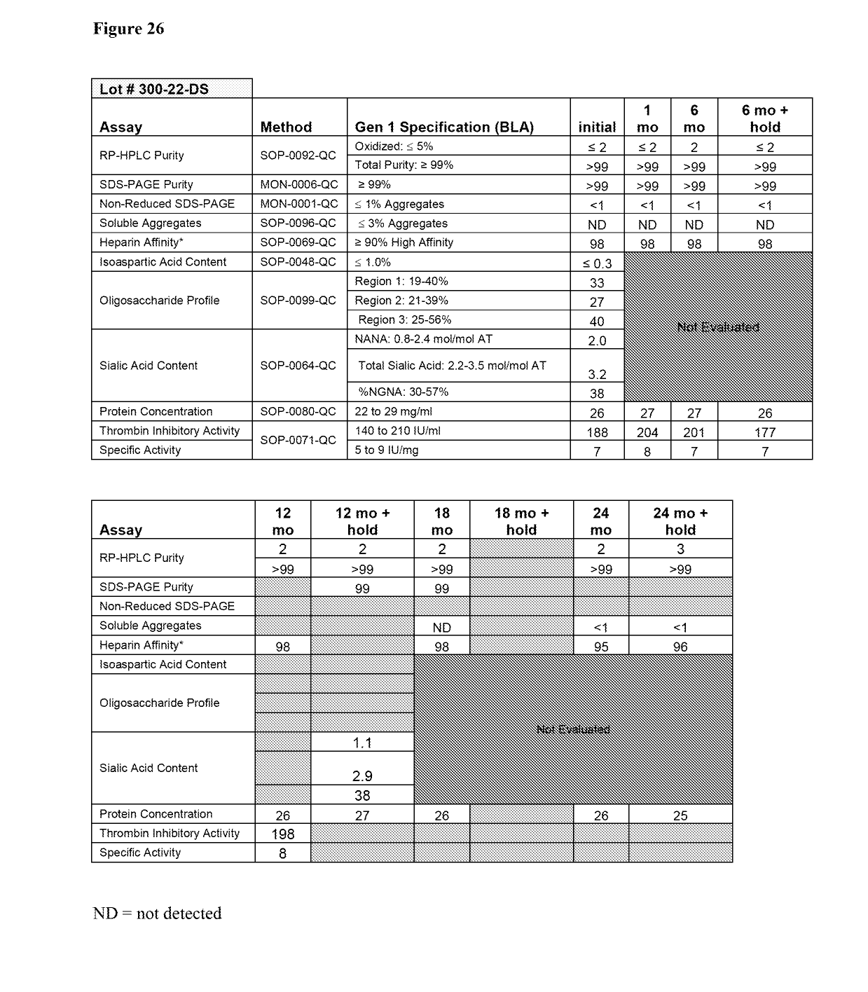

FIG. 27 shows the stability of antithrombin formulation lot #300-23-DS.

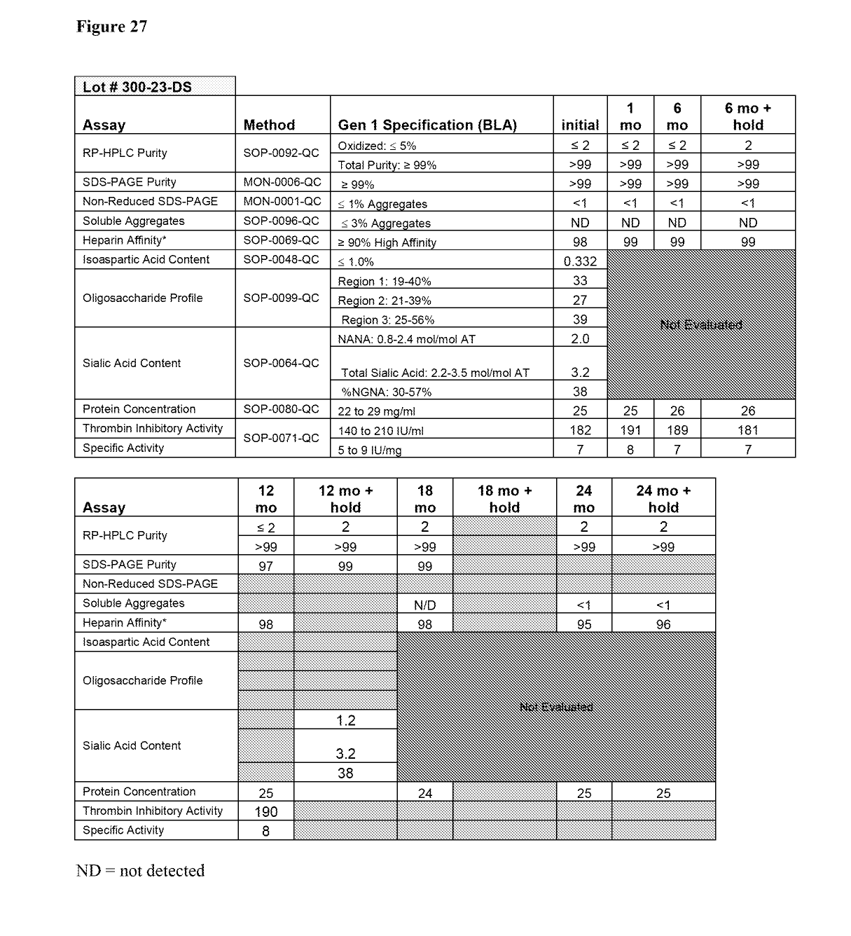

FIG. 28 shows the oxidation of antithrombin formulations.

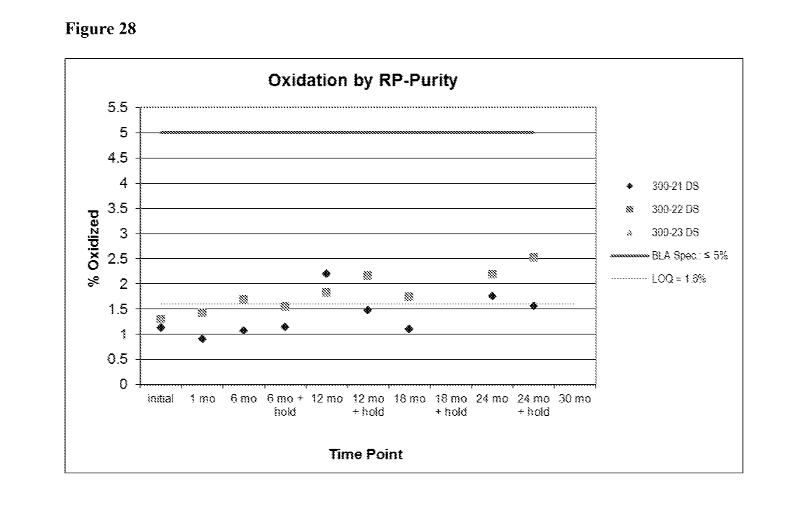

FIG. 29 shows the heparin affinity of antithrombin formulations.

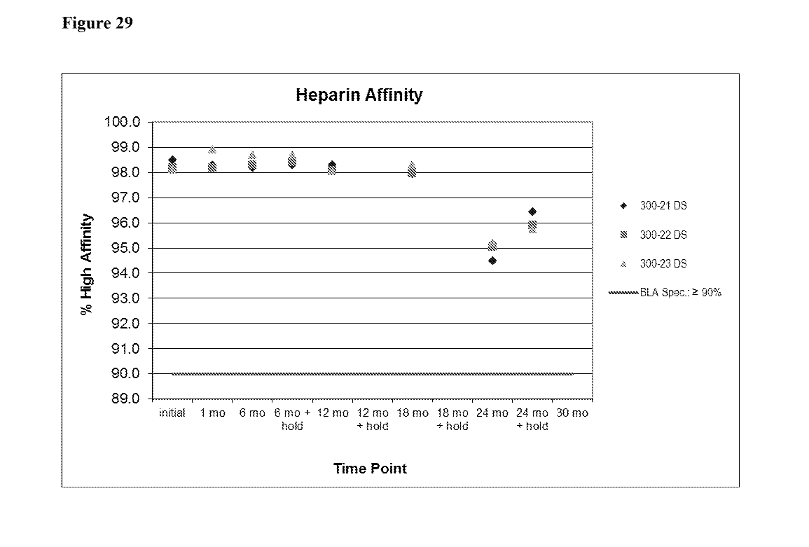

FIG. 30 shows the aggregation of antithrombin formulations.

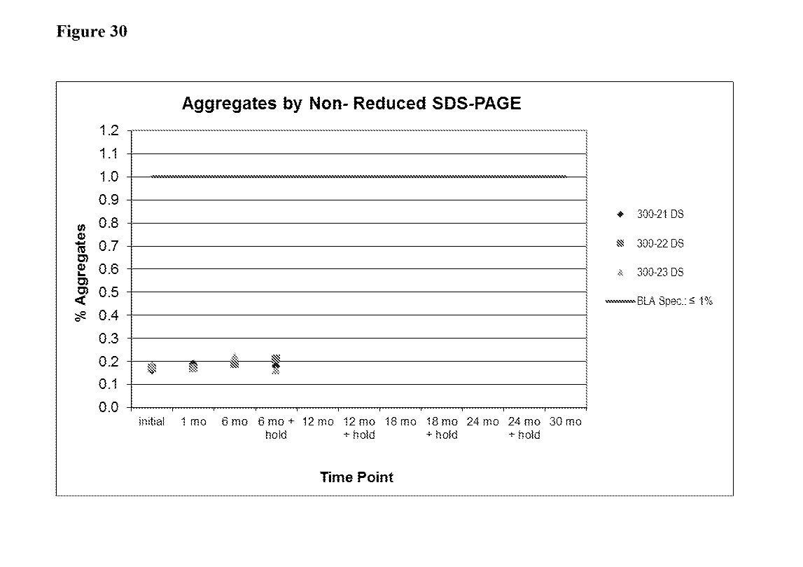

FIG. 31 shows the protein concentration of antithrombin formulations.

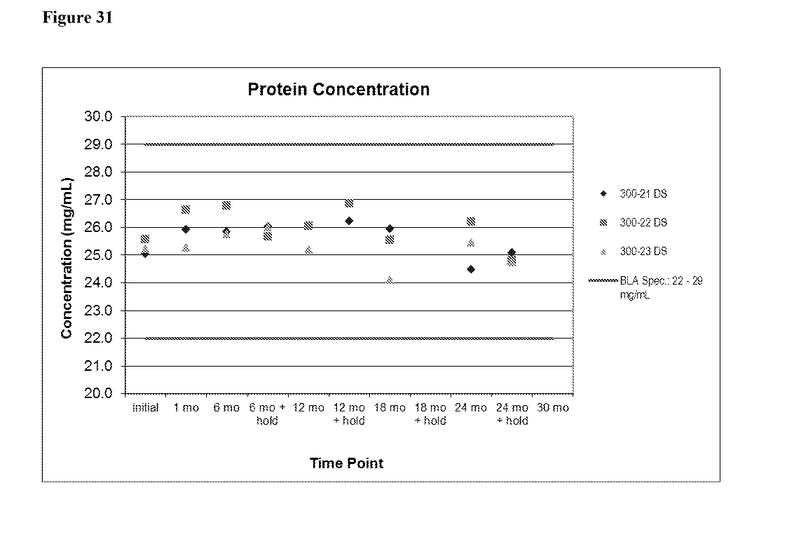

FIG. 32 shows the thrombin inhibitory activity of antithrombin formulations.

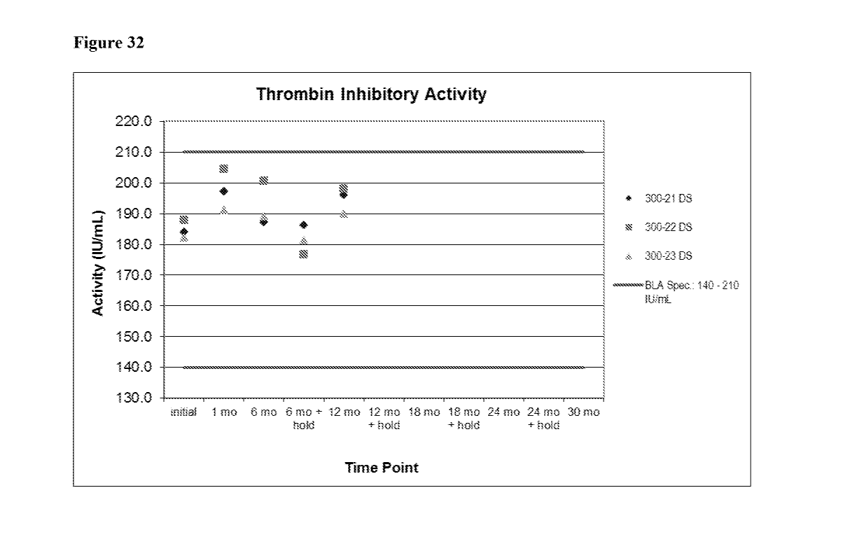

FIG. 33 shows the specific activity of antithrombin formulations.

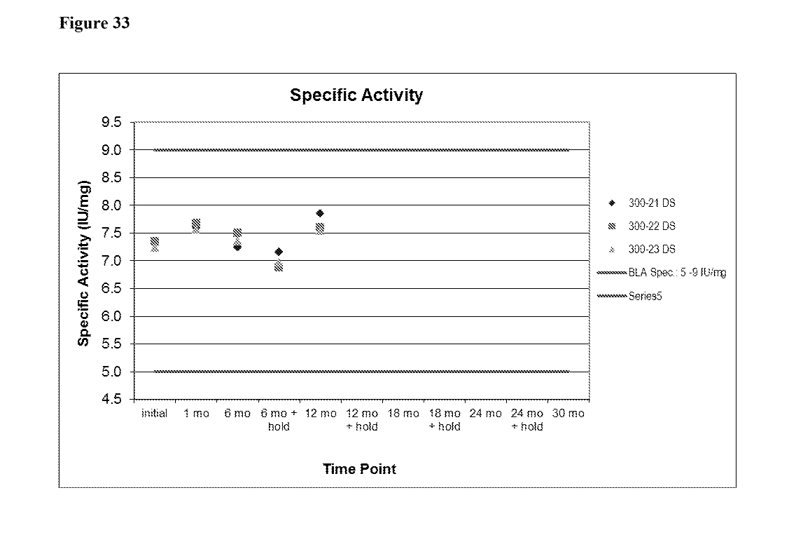

The figures are illustrative only and are not required for enablement of the disclosure.

### DETAILED DESCRIPTION OF THE INVENTION

In one aspect, the disclosure provides formulations that stabilize proteins, such as therapeutic proteins.

Therapeutic proteins, as used herein, are proteins that can be administered to a subject to treat a disease or disorder. Therapeutic proteins include proteins that are produced by living organisms, such as bacteria, plants, yeast, insect cells, mammalian cell lines and transgenic mammals, and proteins that are synthetically produced. Examples of therapeutic proteins include antibodies (e.g., monoclonal antibodies), blood proteins (e.g., factor VIII), enzymes (e.g., alpha galactosidase) and hormones such as insulin. Proteins (and therapeutic proteins), as used herein, also include proteins (and therapeutic proteins) that have been modified (e.g., by glycosylation, or by labeling).

In some embodiments, the formulation comprises a buffer, wherein the buffer comprises mono-hydrogen-phosphate and di-hydrogen-phosphate, wherein the mono-hydrogen-phosphate and di-hydrogen-phosphate have the same counter ion. In some embodiments, the counter ion is sodium or potassium. In some embodiments, the formulation comprises a buffer, wherein the buffer comprises potassium mono-hydrogen-phosphate and potassium di-hydrogen-phosphate. In some embodiments, the formulation comprises a buffer, wherein the buffer comprises sodium mono-hydrogen-phosphate and sodium di-hydrogen-phosphate.

In some embodiments, the formulation comprises a buffer, wherein the buffer essentially consists of potassium mono-hydrogen-phosphate and potassium di-hydrogen-phosphate. In some embodiments, the formulation comprises a buffer, wherein the buffer essentially consists of sodium mono-hydrogen-phosphate and sodium di-hydrogen-phosphate. In some embodiments, the formulation does not include both sodium mono-hydrogen-phosphate and potassium di-hydrogen-phosphate. In some embodiments, the formulation does not include both potassium mono-hydrogen-phosphate and sodium di-hydrogen-phosphate.

In some embodiments, the formulations comprise a protein. In some embodiments, the formulations comprise a therapeutic protein. In some embodiments, the therapeutic protein is antithrombin.

In some embodiments, the disclosure provides formulations that allow for the prolonged storage of proteins without compromising the stability of these proteins. In some embodiments, the formulations disclosed herein allow for prolonged storage of proteins (e.g., therapeutic proteins) at different stages of the production and purification process. In some embodiments, the formulations disclosed herein allow for prolonged storage of proteins (e.g., therapeutic proteins) at elevated temperatures (i.e., −20° C., 4° C., or room temperature), while maintaining the protein stability. In some embodiments, the formulations disclosed herein allow for prolonged storage of proteins (e.g., therapeutic proteins) at lower temperatures (i.e., −40° C. or −60° C.), while maintaining the protein stability. In some embodiments, the formulations disclosed herein maintain the stability of proteins (e.g., therapeutic proteins), even if the storage conditions are not ideal, for instance if the formulation comprising the protein (e.g., therapeutic protein) undergoes a freeze-thaw cycle.

It was surprisingly found herein that formulations that comprise a buffer, wherein the buffer comprises mono-hydrogen-phosphate and di-hydrogen-phosphate, and wherein both phosphate ions have the same counter ion, maintain the stability of proteins. Thus, in one aspect the disclosure provides a formulation comprising a buffer, wherein the buffer comprises mono-hydrogen-phosphate and di-hydrogen-phosphate, and wherein both phosphate ions have the same counter ion. It was also surprisingly found herein that formulations that comprise a buffer, wherein the buffer comprises mono-hydrogen-phosphate and di-hydrogen-phosphate, and wherein both phosphate ions do not have the same counter-ion, do not maintain the stability of proteins.

Formulations comprising a buffer, wherein the buffer comprises potassium mono-hydrogen-phosphate and potassium di-hydrogen-phosphate and formulations comprising a buffer, wherein the buffer comprises sodium mono-hydrogen-phosphate and sodium di-hydrogen-phosphate were found to stabilize therapeutic proteins. In contrast, formulations comprising a buffer, wherein the buffer comprises potassium mono-hydrogen-phosphate and sodium di-hydrogen-phosphate or formulations comprising a buffer, wherein the buffer comprises sodium mono-hydrogen-phosphate and potassium di-hydrogen-phosphate did not stabilize the therapeutic proteins.

In some embodiments, the formulations disclosed herein stabilize a protein. In some embodiments, the formulations disclosed herein stabilize a therapeutic protein. In some embodiments, the therapeutic protein is antithrombin.

The formulations disclosed herein can be used to stabilize proteins regardless of the method of production of the protein (e.g., transgenically, recombinantly or synthetically). In some embodiments, the formulations disclosed herein stabilize milk-produced protein. In some embodiments, the formulations disclosed herein stabilize recombinantly produced protein. In some embodiments, the formulations disclosed herein stabilize milk-produced therapeutic protein. In some embodiments, the formulations disclosed herein stabilize recombinantly produced therapeutic protein. In some embodiments, the formulations disclosed herein stabilize milk-produced antithrombin. In some embodiments, the formulations disclosed stabilize recombinantly produced antithrombin.

The formulations disclosed herein can be used to stabilize proteins during any phase of the production process of the protein. In some embodiments, the formulations disclosed herein are used to stabilize proteins immediately after the harvest stage (e.g., immediately after harvesting the protein from the milk of transgenic animal, immediately after harvesting the protein from lysed cells, or immediately after synthesizing the protein). In some embodiments, the formulation comprises milk. In some embodiments, the formulation comprises components from lysed cells or components from protein synthesis.

In some embodiments, the formulations disclosed herein are used to stabilize proteins that are only partially purified. For instance, the protein may be harvested and undergo one or two purification steps prior to combining the protein with any of the buffers disclosed herein to generate any of the formulations disclosed herein. In some embodiments, the formulation comprises clarified milk product. In some embodiments, the formulation comprises components from a partially purified cell lysate or components from a partially purified protein synthesis reaction. In some embodiments, the formulation includes one or more proteins or polypeptides in addition to the protein (e.g., therapeutic protein) to be stabilized. In some embodiments, the formulation includes non-protein components.

In some embodiments, a composition or solution comprising the protein may undergo multiple purification steps prior to combining the protein with any of the buffers disclosed herein to generate any of the formulations disclosed herein. In some embodiments, a composition or solution comprising the protein is pasteurized prior to combining the protein with any of the buffers disclosed herein (e.g., potassium mono-hydrogen-phosphate and potassium di-hydrogen-phosphate or sodium mono-hydrogen-phosphate and sodium di-hydrogen-phosphate). It should be appreciated that the protein may undergo combinations of pasteurization and purification steps prior to being combined with any of the buffers disclosed herein. Thus, for instance, a protein (e.g., therapeutic protein) can undergo a first purification step, a pasteurization step and a second purification step before protein is combined with any of the buffers disclosed herein.

In some embodiments, the protein is produced in the milk of a transgenic animal and the protein is harvested from the milk of the transgenic animal. In some embodiments, the milk solution is clarified to remove insoluble components. In some embodiments, the milk is clarified by filtration. In some embodiment, no additional purification steps are performed and components are added after these partial purification steps to generate the formulations comprising therapeutic protein disclosed herein. In some embodiments, the formulation comprising therapeutic protein is further purified prior to administration. In some embodiments, the formulation comprising therapeutic protein is shipped prior to further purification for administration. In some embodiment, the formulation comprising therapeutic protein is subjected to nanofiltration e.g., to remove viruses and viral particles, prior to shipment and/or administration.

In some embodiments, the protein formulation is purified to allow for the analysis of the stability of the proteins of the formulation. In some embodiment, the protein is antithrombin and the formulation is purified by contacting the formulation with a heparin column to remove impurities. In some embodiments, the formulation is purified by contacting the formulation with a cation exchange column. In some embodiments, the stability of a protein is analyzed by determining “stability indicators”, e.g., aggregation, oxidation after purifying the formulation. In some embodiments, the protein is antithrombin and the formulation is analyzed by determining “stability indicators”, e.g., aggregation, oxidation after purifying the formulation on a heparin column and a cation exchange column.

The disclosure embraces any method for establishing the formulations comprising a protein disclosed herein. In some embodiments, the protein is added (e.g., as a solid or as concentrate) to any of the buffers described herein to generate the formulations of the disclosure. In some embodiments, the formulation is established by adding one or more buffer components (e.g., a concentration of potassium phosphate) to a composition or solution comprising the protein. In some embodiments, the formulation is established by replacing the buffer of a composition or solution comprising the protein to be stabilized with a buffer of the disclosure (e.g., potassium mono-hydrogen-phosphate and potassium di-hydrogen-phosphate or sodium mono-hydrogen-phosphate and sodium di-hydrogen-phosphate). Replacing a buffer can be done, for instance, by adding a composition or solution comprising the protein to be stabilized to a column resulting in the immobilization of the protein, and eluting the protein with one of the buffers disclosed herein (e.g., potassium mono-hydrogen-phosphate and potassium di-hydrogen-phosphate or sodium mono-hydrogen-phosphate and sodium di-hydrogen-phosphate). Combinations of the above described methods for establishing the formulations described herein are embraced as well.

### Stability

In one aspect, the disclosure provides formulations that stabilize proteins. In some embodiments, the protein is a therapeutic protein. In some embodiments, the therapeutic protein is antithrombin.

A “formulation that stabilizes protein”, as used herein, is a formulation that maintains the stability of a protein over a period of time (e.g., one month or two months), preferably at elevated temperatures (e.g., −20° C. or 4° C.), or after undergoing one or more freeze/thaw cycles.

The formulations disclosed herein stabilize proteins over a period of time. In some embodiments, the formulations stabilize the protein over a period of time more than 1 day, more than 2 days, more than 5 days, more than a week, more than a month, more than 2 months, more than a year, up to 10 years. In some embodiments, the formulation stabilizes the protein for more than a year. In some embodiments, the formulation stabilizes the protein for two years.

In some embodiments, the formulations disclosed herein stabilize proteins at an elevated temperature. In some embodiments, an elevated temperature is more than −60° C., more than −50° C., more than −40° C., more than −30° C., more than −20° C., more than −10° C., more than 0° C., or more than 20° C. In some embodiments, the elevated temperature is −20° C. In still other embodiments, the elevated temperature is in the range of 0° C. to −60° C., 0° C. to −50° C., 0° C. to −40° C., 0° C. to −30° C., 0° C. to −20° C., 0° C. to 20° C., or 2° C. to 8° C. In some embodiments, the elevated temperature is in the range of 2° C. to 8° C. In some embodiments, the formulations disclosed herein stabilize proteins even when the formulation undergoes one or more freeze-thaw cycles. In some embodiments, the formulation stabilizes the protein at −20° C. for two years.

The “stability of a protein” as used herein, refers to the persistence of structural integrity and the functionality of a protein over a period of time. Thus, a protein is stable if the protein maintains its structural integrity and its functionality (e.g., biological functionality) over a specific period of time. Analogously, as described above, a formulation that stabilizes a protein is a formulation that maintains the stability of a protein over a period of time.

The structural integrity of a protein refers to the integrity of the conformation of the protein's polypeptide chain and the integrity of the chemistry of the amino acids and amino acid side chains in the polypeptide chain. A protein that has maintained structural integrity is a protein that has maintained the conformation of the polypeptide chain and the chemistry of the amino acids and amino acid side chains in the polypeptide chain. For instance, a protein has maintained structural integrity over a period of time if the polypeptide has the same conformation after the period of time as compared to before the period of time and if the chemistry of the amino acids and amino acid side chains in the polypeptide chain has not changed during that period of time. It should be appreciated that maintaining the same oligomerization state is also a measure of structural integrity of a protein. Thus, a protein likely has maintained structural integrity if the protein has maintained the same oligomerization state (e.g., has remained a monomer). A person of ordinary skill in the art will know how to determine the structural integrity (conformation, chemistry of amino acids and oligomerization state) of a protein. The conformation of a protein can be determined using standard laboratory techniques including X-ray crystallography, spectroscopy including circular dichroism spectroscopy and fluorescent spectroscopy, and nuclear magnetic resonance. The chemistry of the amino acids, including the chemistry of the side chains, can be determined by chemical reactions to test for the presence of specific chemical groups (for instance, determining the oxidation state of the side chains), or by the above described laboratory techniques that can determine the structure of the protein. The oligomerization state of a protein can be determined for instance by size exclusion chromatography (SEC).

The functionality of a protein refers to the function (e.g., the biological function) the protein performs. A protein that has maintained functionality is protein that has maintained its ability to perform a specific (biological) function. For instance, a protein has maintained functionality over a period of time if the protein has the same ability to perform a specific function as compared to the ability prior to the period of time. Examples of functionality include the ability to perform an enzymatic reaction (e.g., cleave a peptide bond), bind a target (e.g., block a receptor) or illicit a cellular response (e.g., by activating a receptor). The specific method for determining the functionality of each protein will depend on the nature of the protein. A person of ordinary skill in the art can use methods known in the art to find which functional assay is needed to determine the functional activity of a specific protein. Many of the functionalities of a protein require binding of the protein to a target. Thus, the functionality of a protein can often be determined by investigating if a protein can bind a particular target. This binding can be determined in a structural assay (is there binding) or a functional assay (can the protein perform its biological function, e.g., can it initiate a cell signaling cascade, can it perform an enzymatic function, can it block a protein-protein interaction). Examples of functional assays are binding assays, enzymatic assays and cellular assays.

In some embodiments, the stability of a protein is determined by comparing the structural integrity and/or functionality of the protein at the beginning of a period of time to the structural integrity and/or functionality of the protein at the end of a period of time (e.g., a three month period). For instance, the percentage of aggregation of a protein is determined prior to a specific period of time and compared to the percentage of aggregation of the protein after the period of time.

In some embodiments, the stability of a protein is determined by comparing the structural integrity and/or functionality of the protein at different storage conditions. For instance, the percentage of aggregation of a protein is determined in a first aliquot that has been stored at between 2-8° C. over a specific period of time, and compared to a second aliquot that has been stored at −20° C. over the same period of time.

In some embodiments, the stability of a protein is determined by determining the absolute value of the structural integrity and/or functionality of the protein without comparison to a different condition, time point. For instance, the percentage of aggregation of a protein is determined after a specific period of time and compared to a predetermined standard. For instance, in some embodiments, a protein is considered to be stable if less than 5% of the protein in a specific sample is aggregated. In some embodiments, a protein formulation is considered stable if the percentage of aggregation of the protein in the formulation is low enough to allow for nanofiltration of the formulation. In some embodiments, nanofiltration is used as a test to determine if the protein formulation is acceptable for shipment and/or administration: if the formulation can be run through a nanofilter, the formulation is acceptable for shipment.

In some embodiments, protein stability is determined by comparing the structural integrity and/or functionality of the protein prior to and after the period of time (e.g., one month, two months, or three months). In some embodiments, a protein is stabilized if more than 50%, more than 60%, more than 70%, more than 80%, more than 90%, more than 91%, etc. up to more than 99% of the structural integrity and/or functionality is maintained after a period of time when compared to the structural integrity and/or functionality prior to that period of time. For instance, in some embodiments, a protein is stabilized if more than 95% of the functionality of the protein is maintained when the protein has been stored three months compared to the functionality prior to storage.

In some embodiments, protein stability is determined by comparing structural integrity and/or functionality when a protein is stored at different temperatures for a period of time (e.g., one month, two months, or three months). In some embodiments, a protein is stabilized if more than 50%, more than 60%, more than 70%, more than 80%, more than 90%, more than 91%, etc. up to more than 99% of structural integrity and/or functionality is maintained when the protein is stored at an elevated temperature compared to storage at a lower temperature. For instance, in some embodiments, a protein is stabilized if more than 95% of the functionality of the protein is maintained when the protein is stored at an elevated temperature (e.g., between 2° C.-8° C.) as compared to storage at a lower temperature (e.g., −20° C.).

In some embodiments, the protein whose stability is to be determined is a therapeutic protein. In some embodiments, the therapeutic protein is antithrombin. In some embodiments the stability of antithrombin is determined by determining the percentage or amount of antithrombin that can bind heparin. In some embodiments the stability of antithrombin is determined by determining the percentage, or amount (by weight), of antithrombin that is aggregated. In some embodiments the stability of antithrombin is determined by determining the percentage of antithrombin that has been oxidized.

In some embodiments, the stability of antithrombin is determined by comparing the ability to bind heparin prior to and after storage for a period of time. In some embodiments, stability of antithrombin is determined by comparing the ability to bind heparin in a first aliquot that is stored at an increased temperature compared to an aliquot that is stored at a lower temperature for the same period of time. In some embodiments, antithrombin is stabilized if more than 50%, more than 60%, more than 70%, more than 80%, more than 90%, more than 91%, etc. up to more than 99% of antithrombin can bind heparin after storage as compared to prior to storage for a period of time. In some embodiments, antithrombin is stabilized if more than 50%, more than 60%, more than 70%, more than 80%, more than 90%, more than 91%, etc. up to more than 99% of antithrombin can bind heparin after storage at an increased temperature compared to an aliquot that is stored at a lower temperature for the same period of time. In some embodiments, antithrombin is stabilized if more than 95% of antithrombin can bind heparin when antithrombin is stored at an elevated temperature (e.g., between 2° C.-8° C.) compared to a lower temperature (e.g., −20° C.) for a period of time (e.g., three months).

In some embodiments, the stability of antithrombin is determined by comparing the aggregation (by weight) of antithrombin prior to and after storage for a period of time. In some embodiments, the stability of antithrombin is determined by comparing the aggregation (by weight) of antithrombin in first aliquot that is stored at an increased temperature compared to an aliquot that is stored at a lower temperature for the same period of time. In some embodiments, antithrombin is stabilized if less than 10 times, less than 9 times, less than 8 times, less than 7 times, less than 6 times, less than 5 times, less than 4 times, less than 3 times, less than 2 times, less than 1.5 times and up to the same amount of antithrombin is in an aggregated form after storage when compared to the amount of aggregation prior to storage. In some embodiments, antithrombin is stabilized if less than 10 times, less than 9 times, less than 8 times, less than 7 times, less than 6 times, less than 5 times, less than 4 times, less than 3 times, less than 2 times, less than 1.5 times and up to the same amount of antithrombin is in an aggregated form when antithrombin is stored at an elevated temperature as compared to a lower temperature. In some embodiments, antithrombin is stabilized if less than 3 times the amount of antithrombin is in an aggregated form (by weight) when a protein is stored at an elevated temperature (e.g., between 2° C.-8° C.) as compared to a lower temperature (e.g., −20° C.) for a period of time (e.g., three months).

In some embodiments, the stability of antithrombin is determined by comparing the oxidation of antithrombin prior to and after storage for a period of time. In some embodiments, the stability of antithrombin is determined by comparing the oxidation of antithrombin in a first aliquot that is stored at an increased temperature compared to an aliquot that is stored at a lower temperature for the same period of time. In some embodiments, antithrombin is stabilized if less than 10 times, less than 9 times, less than 8 times, less than 7 times, less than 6 times, less than 5 times, less than 4 times, less than 3 times, less than 2 times, less than 1.5 times and up to the same amount of antithrombin is oxidized after storage when compared to the amount of aggregation prior to storage. In some embodiments, antithrombin is stabilized if less than 10 times, less than 9 times, less than 8 times, less than 7 times, less than 6 times, less than 5 times, less than 4 times, less than 3 times, less than 2 times, less than 1.5 times and up to the same amount of antithrombin is oxidized when antithrombin is stored at an elevated temperature as compared to a lower temperature. In some embodiments, antithrombin is stabilized if less than 2 times the amount of antithrombin is oxidized when a protein is stored at an elevated temperature (e.g., between 2° C.-8° C.) compared to a lower temperature (e.g., −20° C.) for a period of time (e.g., three months).

In some embodiments, antithrombin is stabilized if at least 90% of antithrombin binds heparin after three months of storage as compared to prior to storage, or if less than 2% of antithrombin is oxidized, or if less than 5% of antithrombin is aggregated, or if at least 90% of the antithrombin binds heparin.

In some embodiments, antithrombin is stabilized if at least 90% of antithrombin binds heparin after three months of storage as compared to prior to storage, and less than 2% of antithrombin is oxidized, and less than 5% of antithrombin is aggregated, and at least 90% of the antithrombin binds heparin.

### Formulation

In some embodiments, the formulation comprises a buffer. A buffer as used herein is a composition comprising a weak acid and its conjugate base or a combination of a weak base and its conjugate acid. Compositions or solutions comprising a buffer generally have a more stabilized pH than compositions or solutions without a buffer.

In some embodiments, the formulation comprises a buffer, wherein the buffer comprises mono-hydrogen-phosphate and di-hydrogen-phosphate, wherein the mono-hydrogen-phosphate and di-hydrogen-phosphate have the same counter ion. In some embodiments, the counter ion is sodium or potassium. In some embodiments, the formulation comprises a buffer, wherein the buffer comprises potassium mono-hydrogen-phosphate and potassium di-hydrogen-phosphate. In some embodiments, the formulation comprises a buffer, wherein the buffer comprises sodium mono-hydrogen-phosphate and sodium di-hydrogen-phosphate.

In some embodiments, the formulation comprises a buffer, wherein the buffer essentially consists of potassium mono-hydrogen-phosphate and potassium di-hydrogen-phosphate. In some embodiments, the formulation comprises a buffer, wherein the buffer essentially consists of sodium mono-hydrogen-phosphate and sodium di-hydrogen-phosphate. In some embodiments, the formulation does not include both sodium mono-hydrogen-phosphate and potassium di-hydrogen-phosphate. In some embodiments, the formulation does not include both potassium mono-hydrogen-phosphate and sodium di-hydrogen-phosphate.

A buffer that “essentially consists of” potassium mono-hydrogen-phosphate and potassium di-hydrogen-phosphate is a buffer that in addition to potassium mono-hydrogen-phosphate and potassium di-hydrogen-phosphate does not have similar amounts of additional ions or other components that can act as a buffer. Similar amounts, as us herein refers, to an amount that is the same, 0.9 times the amount, 0.8 times the amount, 0.7 times the amount, 0.6 times the amount, 0.5 times the amount, 0.4 times the amount, 0.3 times the amount, up to 0.2 times the amount. Thus, for instance, a buffer that essentially consists of potassium mono-hydrogen-phosphate and potassium di-hydrogen-phosphate and that includes 50 mM potassium mono-hydrogen-phosphate and potassium di-hydrogen-phosphate, will not also include 50 mM sodium mono-hydrogen-phosphate or 50 mM sodium di-hydrogen-phosphate.

Analogously, a buffer that “essentially consists of” sodium mono-hydrogen-phosphate and sodium di-hydrogen-phosphate is a buffer that in addition to sodium mono-hydrogen-phosphate and sodium di-hydrogen-phosphate does not have similar amounts of additional ions or other components that can act as a buffer.

Thus, a buffer that “essentially consists of” potassium mono-hydrogen-phosphate and potassium di-hydrogen-phosphate, will not also include similar amounts of non-potassium (e.g., sodium) mono-hydrogen-phosphate and non-potassium (e.g., sodium) di-hydrogen-phosphate. Analogously, a buffer that “essentially consists of” sodium mono-hydrogen-phosphate and sodium di-hydrogen-phosphate, will not also include similar amounts of non-sodium (e.g., potassium) mono-hydrogen-phosphate and non-sodium (e.g., potassium) di-hydrogen-phosphate.

In some embodiments, the buffer concentration is between 10 mM and 250 mM or between 25 mM and 100 mM. In some embodiments, the buffer concentration is 50 mM.

In some embodiments, the formulation further includes one or more salts. In some embodiments, the salt is potassium chloride. In some embodiments, the potassium chloride concentration is between 1 mM and 250 mM, between 2 mM and 200 mM, or between 10 and 150 mM. In some embodiments, the potassium chloride concentration is 120 mM.

In some embodiments, the formulation includes one or more salts in addition to potassium chloride. Non-limiting examples of salts that can be used in the formulations include ammonium salts and calcium salts. In some embodiments, the concentration of these one or more additional salts is between 10 mM and 250 mM, between 25 mM and 100 mM. In some embodiments, the salt concentration is 50 mM. In some embodiments, the salt concentration is less than 10 mM. In some embodiments, the salt concentration is more than 250 mM. In some embodiments, the salt concentration is 50 mM.

In some embodiments, the formulation includes therapeutic protein. In some embodiments, the therapeutic protein is albumin, alpha-macroglobulin, antichymotrypsin, antithrombin, antitrypsin, Apo A, Apo B, Apo C, Apo D, Apo E, Apo F, Apo G, beta XIIa, C1-inhibitor, C-reactive protein, C7 protein, C1r protein, C1s protein, C2 protein, C3 protein, C4 protein, C4bP protein, C5 protein, C6 protein, C1q protein, C8 protein, C9 protein, carboxypeptidase N, ceruloplasm, Factor B, Factor D, Factor H, Factor I, Factor IX, Factor V, Factor VII, Factor VIIa, Factor VIII, Factor X, Factor XI, Factor XII, Factor XIII, fibrinogen, fibronectin, haptoglobin, hemopexin, heparin cofactor II, histidine-rich GP, IgA, IgD, IgE, IgG, ITI, IgM, kininase II, kininogen, lysozyme, PAI 2, PAI 1, PCI, plasmin, plasmin inhibitor, plasminogen, prealbumin, prokallikrein, properdin, protease nexin, Protein C, Protein S, Protein Z, prothrombin, TFPI, thiol-proteinase, thrombomodulin, tissue factor (TF), TPA, transcolabamin II, transcortin, transferrin, vitronectin, or von Willebrand factor.

In some embodiments, the formulation includes 1 to 50 mg/ml of therapeutic protein, 2 to 25 mg/ml of therapeutic protein, 3 to 10 mg/ml of therapeutic protein, 4 to 8 mg/ml of therapeutic protein or 5 to 6 mg/ml of therapeutic protein. In some embodiments, the formulation includes less than 1 mg/ml of therapeutic protein. In some embodiments, the formulation includes more than 50 mg/ml of therapeutic protein.

In some embodiments, the therapeutic protein is antithrombin. In some embodiments, the formulation includes 1 to 50 mg/ml of antithrombin, 2 to 25 mg/ml of antithrombin, 3 to 10 mg/ml of antithrombin, 4 to 8 mg/ml of antithrombin or 5 to 6 mg/ml of antithrombin. In some embodiments, the formulation includes 5 to 6 mg/ml of antithrombin. In some embodiments, the formulation includes less than 1 mg/ml of antithrombin. In some embodiments, the formulation includes more than 50 mg/ml of antithrombin. In some embodiments, the formulation includes up to 100 mg/ml of antithrombin. In some embodiments, the formulation includes more than 100 mg/ml of antithrombin.

It should be appreciated that the formulation can also include additional components, including additional proteins. For instance, a newly harvested solution of therapeutic protein (e.g., not yet, or only partially purified) may include other protein in addition to the therapeutic protein (e.g., milk proteins or proteins found in cell lysate). In some embodiments, the formulation includes a variety of additional non-protein components (e.g., non-protein components found in milk or cell lysate).

In some embodiments, the pH of the formulation is between pH 6 and pH 9, or between pH 7.5 and pH 8.5. In some embodiments, the pH of the formulation is pH 8. If needed, acid (such as HCl) or base (such as NaOH) can be added to a formulation to attain the desired pH.

In some embodiments, the therapeutic protein is antithrombin and the pH of the formulation is between pH 7.5 and pH 8.5. In some embodiments, the therapeutic protein is antithrombin and the pH of the formulation is pH 8. It should be appreciated that the pH of the formulation may depend on the nature of the therapeutic protein.

In some embodiments, the formulation does not contain a stabilizing excipient.

In some embodiments, the formulation includes a stabilizing excipient, such as carboxylic acid or a salt thereof. In some embodiments, the carboxylic acid is sodium citrate. In some embodiments, the formulation includes a monocarboxylic acid and/or salt thereof. In some embodiments, the formulation includes a gluconic acid and/or sodium gluconate. In some embodiments, the formulation includes a dicarboxylic acid and/or a salt thereof. In some embodiments, the formulation includes a citric acid, succinic acid, malonic acid, maleic acid, tartaric acid and or a salt thereof. In some embodiments, the formulation includes a tricarboxylic aid and/or a salt thereof. In some embodiments, the formulation includes a nitrilotriacetic acid and/or sodium nitrilotriacetic acid. In some embodiments, the formulation includes a tetracarboxylic acid and/or salt thereof. In some embodiments, the formulation includes an ethylenediaminetetracetic acid (EDTA) and/or sodium EDTA. In some embodiments, the formulation includes a pentacarboxylic acid and/or a salt thereof. In some embodiments, the formulation includes a diethylenetriaminepentaacetic (DTPA) acid and/or sodium DTPA. Suitable carboxylic acids include, but are not limited to, citrate compounds, such as sodium citrate; tartrate compounds, succinate compounds, malonate, gluconate, 1,2,3,4-Butanetetracarboxylic acid (BTC), EDTA or DTPA or a salt thereof. Kaushil et al. in Protein Science 1999 8: 222-233 and Busby et al. in the Journal of Biological Chemistry Volume 256, Number 23 pages 12140-1210-12147 describe carboxylic acids and their uses. In some embodiments, the stabilizing excipient does not function as a buffer.

In some embodiments, the stabilizing excipient has a concentration of between 50 to 600 mM, between 250 to 500 mM, or between 250 to 350 mM. In some embodiments, the stabilizing excipient is at a concentration of 50 to 100 mM, 50 to 150 mM, 50 to 200 mM, 50 to 250 mM, 50 to 300 mM, 50 to 350 mM, 50 to 400 mM, 50 to 450 mM, 50 to 500 mM or 50 to 550 mM. In some embodiments, the stabilizing excipient is at a concentration of 550 to 600 mM, 500 to 600 mM, 450 to 600 mM, 400 to 600 mM, 350 to 600 mM, 300 to 600 mM, 250 to 600 mM, 200 to 650 mM, 150 to 600 mM or 100 to 600 mM. In some embodiments, the stabilizing excipient is at a concentration of 100 to 550 mM, 150 to 500 mM, 200 to 450 mM, 250 to 400 mM or 300 to 350 mM. In some embodiments, the stabilizing excipient is at a concentration of 100, 150, 250, 500 or 600 mM. In some embodiments, the concentration of the stabilizing excipient is less than 100 mM. In some embodiments, the concentration of the stabilizing excipient is more than 600 mM. In one embodiment, the stabilizing excipient is at a concentration of 300 mM.

In some embodiments, the formulation includes a sugar (e.g., a disaccharide sugar). In general, the sugars may have an additional stabilizing effect and can minimize aggregation of proteins. In some embodiments, the sugar is a disaccharide sugar. Disaccharide sugars that can be added to the formulation include, but are not limited to, sucrose, lactulose, lactose, maltose, trehalose and cellobiose. In some embodiments, the formulation includes sucrose or trehalose as the disaccharide.

In some embodiments, the sugar is present at between 0.5 to 5% (wt/volume). In some embodiments, the sugar is at least 0.5%, at least 1%, at least 1.5%, at least 2%, at least 2.5%, at least 3%, at least 3.5%, at least 4%, at least 4.5%, or up to 5% of volume by weight. In some embodiments, the sugar is present at between 1 to 2% (wt/volume). In some embodiments, the sugar is present at 1% (wt/volume). In some embodiments, the sugar is present at less than 1% (wt/volume). In some embodiments, the sugar is present at more than 5% (wt/volume). In one embodiment, the sugar is sucrose or trehalose and is present at 1% (wt/volume).

In some embodiments, the stable liquid formulation does not include a surfactant. In some embodiments, the stable liquid formulation further comprises one or more surfactants. In some embodiments, the surfactant is Polysorbate 80, Polysorbate 20, Tween 20 or Tween 80. In some embodiments, the surfactant is 0.5 to 1% of volume by volume. In some embodiments, the surfactant is 0.5 or 1% of volume by volume. In some embodiments, the surfactant has little (e.g., less than 5 mM, less than 4 mM, less than 3 mM, less than 2 mM or less than 1 mM hydrogen peroxide) or no hydrogen peroxide contamination.

In some embodiments, the formulation of therapeutic protein is contained in a syringe, vial, bottle, ampoule or bag. In some embodiments, the bag is an EVA bag. In another embodiment, the bottle is a PETG bottle.

In some embodiments, the formulation comprises 50 mM potassium phosphate, (potassium mono-hydrogen-phosphate and potassium di-hydrogen-phosphate), and 120 mM potassium chloride and the pH=8. In some embodiments, the formulation essentially consists of 50 mM potassium phosphate, (potassium mono-hydrogen-phosphate and potassium di-hydrogen-phosphate), and 120 mM potassium chloride and the pH=8.

### Antithrombin

In some embodiments, the formulation comprises a therapeutic protein. In some embodiments, the therapeutic protein is antithrombin. Antithrombin is generally a glycoprotein of 432 amino acids and a molecular weight of 58 kDA that is a serine protease inhibitor that inhibits thrombin and Factor Xa. The antithrombin can be the alfa (or alpha) form of Antithrombin III, but the formulations of the disclosure can be used for any form of antithrombin. Antithrombin is naturally present in plasma, and human antithrombin may be isolated from human plasma. Human antithrombin may also be produced by recombinant methods, resulting in recombinant human antithrombin (rhAT; unless specifically stated the term “antithrombin”, as used herein, includes rhAT).

Recombinant antithrombin alfa can be produced in transgenic animals and can be used to treat subjects deficient in antithrombin alfa (See e.g., U.S. Pat. No. 5,843,705, U.S. Pat. No. 6,441,145 and U.S. Pat. No. 7,019,193). ATryn® is a recombinantly produced human antithrombin alfa that is approved by the FDA for the prevention of peri-operative and peri-partum thromboembolic events in hereditary antithrombin deficient patients. In Europe, ATryn® is approved for use in surgical patients with congenital antithrombin deficiency for the prophylaxis of deep vein thrombosis and thromboembolism in clinical risk situations. The term “antithrombin”, as used herein, includes ATryn®.

The antithrombin formulations disclosed herein are stable under storage conditions, such as at elevated temperatures. It was found that the formulations of antithrombin disclosed herein have a long shelf-life and maintain the desired level of activity under such storage conditions.

It should be appreciated that the formulations disclosed herein may be used to stabilize formulations of antithrombin that need to processed further prior to administration and formulations that are ready for administration. Thus, in some embodiments, the formulations of antithrombin may be shipped, further processed, purified and/or divided in batches prior to being administered. In some embodiments, the formulations comprise milk-produced antithrombin. In some embodiments, the formulations include antithrombin that has been purified by depth filtration (U.S. Pat. No. 7,531,632) and/or that has been purified by TFF buffer exchange (U.S. Pat. No. 6,268,487). In some embodiments, the antithrombin formulation also contains milk components. In some embodiments the antithrombin formulation has been pasteurized.

In one aspect, the disclosure provides a method for generating a formulation that stabilizes antithrombin, the method comprising separating antithrombin from a milk composition comprising antithrombin resulting in a solution comprising antithrombin, pasteurizing the solution comprising antithrombin, exchanging the solution comprising antithrombin for a buffer,

wherein the buffer comprises potassium mono-hydrogen-phosphate and potassium di-hydrogen-phosphate, or wherein the buffer comprises sodium mono-hydrogen-phosphate and sodium di-hydrogen-phosphate, thereby generating a formulation that stabilizes antithrombin.

### Additives to Formulations

In some embodiments, the formulation includes one or more antioxidants. Antioxidants are substances capable of inhibiting oxidation by removing free radicals from solution. Antioxidants are well known to those of ordinary skill in the art and include materials such as ascorbic acid, ascorbic acid derivatives (e.g., ascorbylpalmitate, ascorbylstearate, sodium ascorbate, calcium ascorbate, etc.), butylated hydroxy anisole, buylated hydroxy toluene, alkylgallate, sodium meta-bisulfite, sodium bisulfite, sodium dithionite, sodium thioglycollic acid, sodium formaldehyde sulfoxylate, tocopherol and derivatives thereof, (d-alpha tocopherol, d-alpha tocopherol acetate, dl-alpha tocopherol acetate, d-alpha tocopherol succinate, beta tocopherol, delta tocopherol, gamma tocopherol, and d-alpha tocopherol polyoxyethylene glycol 1000 succinate) monothioglycerol and sodium sulfite. Such materials are typically added in ranges from 0.01 to 2.0% (wt/volume).

In some embodiments, the formulation includes one or more isotonicity agents. This term is used in the art interchangeably with iso-osmotic agent, and is known as a compound which is added to the pharmaceutical preparation to increase the osmotic pressure to that of 0.9% sodium chloride solution, which is iso-osmotic with human extracellular fluids, such as plasma. Preferred isotonicity agents are sodium chloride, mannitol, sorbitol, lactose, dextrose and glycerol.

In some embodiments, the formulation includes one or more preservatives. Suitable preservatives include but are not limited to: chlorobutanol (0.3-0.9% W/V), parabens (0.01-5.0%), thimerosal (0.004-0.2%), benzyl alcohol (0.5-5%), phenol (0.1-1.0%), and the like (wt/volume).

### Methods

In one aspect the disclosure provides methods for generating formulations that stabilize therapeutic proteins. In some embodiments, the method comprises adding a buffer to a solution followed by the addition of a protein. In some embodiments, the method comprises adding a protein to a solution followed by the addition of a buffer. In some embodiments, the method comprises providing a solution comprising protein and adding a buffer to the solution.

In some embodiments, the method comprises adding a buffer comprising potassium mono-hydrogen-phosphate and potassium di-hydrogen-phosphate. In some embodiments, the method comprises adding a buffer comprising sodium mono-hydrogen-phosphate and sodium di-hydrogen-phosphate. In some embodiments, the method comprises providing a solution comprising buffer and adding protein to the solution. In some embodiments, the buffer comprises potassium mono-hydrogen-phosphate and potassium di-hydrogen-phosphate. In some embodiments, the buffer comprises sodium mono-hydrogen-phosphate and sodium di-hydrogen-phosphate.

In some embodiments, the method comprises removing a buffer from a solution. In some embodiments, the method comprises removing a buffer from a solution and adding a buffer comprising potassium mono-hydrogen-phosphate and potassium di-hydrogen-phosphate. In some embodiments, the method comprises removing a buffer from a solution and adding a buffer comprising sodium mono-hydrogen-phosphate and sodium di-hydrogen-phosphate. In some embodiments, the solution comprises a therapeutic protein. In some embodiments, the adding of buffer and the removing of buffer is done simultaneously. In some embodiments, the adding of buffer and the removing of buffer is done sequentially. The adding and removing of buffer can be done on a solution that comprises a therapeutic protein or the therapeutic protein can be added after the buffers have been exchanged.

In some embodiments, in all of the above methods, the buffer is brought to a concentration level as provided above (e.g., 50 mM). In some embodiments of these methods, the formulation is at or brought to a pH as provided above (e.g., a pH of 8).

Methods for removing and adding a salt to a solution are known in the art and include dialysis, buffer exchange, column purification etc.

### Administration

In some embodiments, the disclosure provides formulations of therapeutic proteins that require further processing prior to administration. In some embodiments, the disclosure provides formulations of therapeutic proteins that are ready for administration. Ready for administration includes formulations that require a minimal step such as thawing and/or transfer to a syringe prior to administration. In some embodiments, the formulations of the present disclosure are intended as a concentrated dosage for intravenous, intra-arterial or parenteral administration. In some embodiments, the formulations, therefore, are also primarily intended as a concentrated dosage for injection.

The formulations described herein, when used in alone or in combination, can be administered in therapeutically effective amounts. A therapeutically effective amount will be determined by the parameters discussed below; but, in any event, is that amount which establishes a level of the drug(s) effective for treating a subject, such as a human subject, having one of the conditions described herein (e.g., hereditary or acquired antithrombin deficiency). An effective amount means that amount alone or with multiple doses, necessary to delay the onset of, inhibit completely or lessen the progression of or halt altogether the onset or progression of the condition being treated. When administered to a subject, effective amounts will depend, of course, on the particular condition being treated; the severity of the condition; individual patient parameters including age, physical condition, size and weight; concurrent treatment; frequency of treatment; and the mode of administration. These factors are well known to those of ordinary skill in the art and can be addressed with no more than routine experimentation. It is preferred generally that a maximum dose be used, that is, the highest safe dose according to sound medical judgment.

The formulations described herein may include or be diluted into a pharmaceutically-acceptable carrier. The term “pharmaceutically-acceptable carrier” as used herein means one or more compatible solid, or semi-solid or liquid fillers, diluants or encapsulating substances which are suitable for administration to a human or other mammal such as a dog, cat, horse, cow, sheep, or goat. The term “carrier” denotes an organic or inorganic ingredient, natural or synthetic, with which the active ingredient is combined to facilitate the application. The carriers are capable of being commingled with the preparations of the present invention, and with each other, in a manner such that there is no interaction which would substantially impair the desired pharmaceutical efficacy or stability. Carriers suitable for intravenous, intra-arterial or parenteral, etc. formulations can be found in Remington's Pharmaceutical Sciences, Mack Publishing Company, Easton, Pa.

In one embodiment, the formulation of therapeutic protein is sterile.

In still another embodiment, the formulation of therapeutic protein is contained in a kit. In one embodiment, the kit further comprises instructions for using the formulation. In another embodiment, the kit further comprises a syringe. In yet another embodiment, such a kit further comprises instructions for administering the formulation. In a further embodiment, the kit further comprises a solution for diluting the formulation. In still another embodiment, such a kit further comprises instructions for mixing the solution for diluting the formulation and the formulation. The aforementioned kits are also provided in another aspect of the invention.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference, in particular for the teaching that is referenced hereinabove. However, the citation of any reference is not intended to be an admission that the reference is prior art.

### EXAMPLES

In the Examples, “K/Na phosphate” refers to potassium mono-hydrogen-phosphate and sodium di-hydrogen-phosphate; “Na/K phosphate” refers to sodium mono-hydrogen-phosphate and potassium di-hydrogen-phosphate; “Na/Na phosphate” refers to sodium mono-hydrogen-phosphate and sodium di-hydrogen-phosphate; “K/K phosphate” refers to potassium mono-hydrogen-phosphate and potassium di-hydrogen-phosphate. These four buffers are collectively referred to herein as the “phosphate systems”.

### Example 1

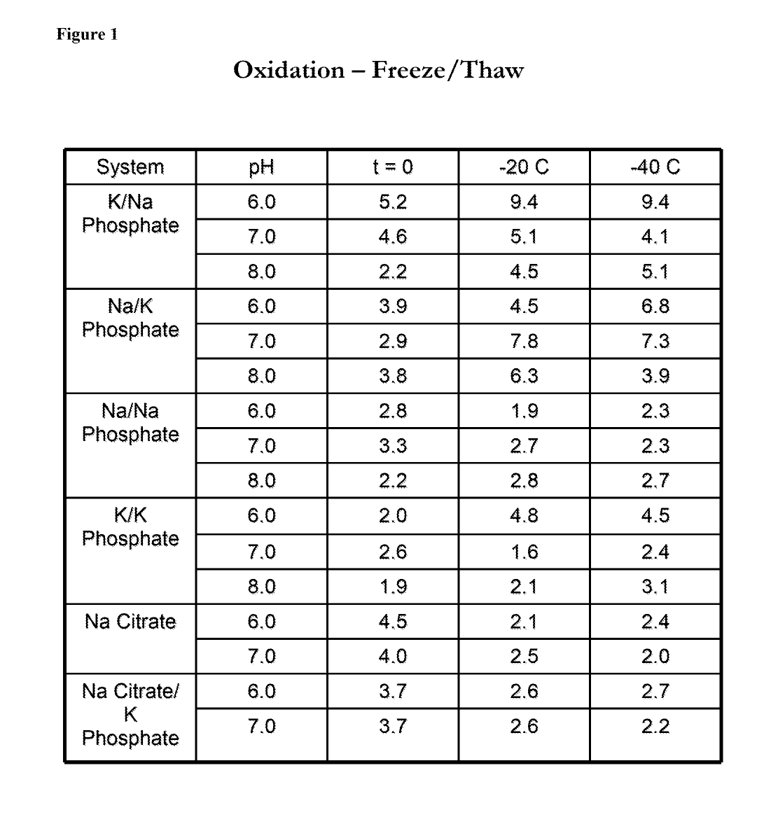

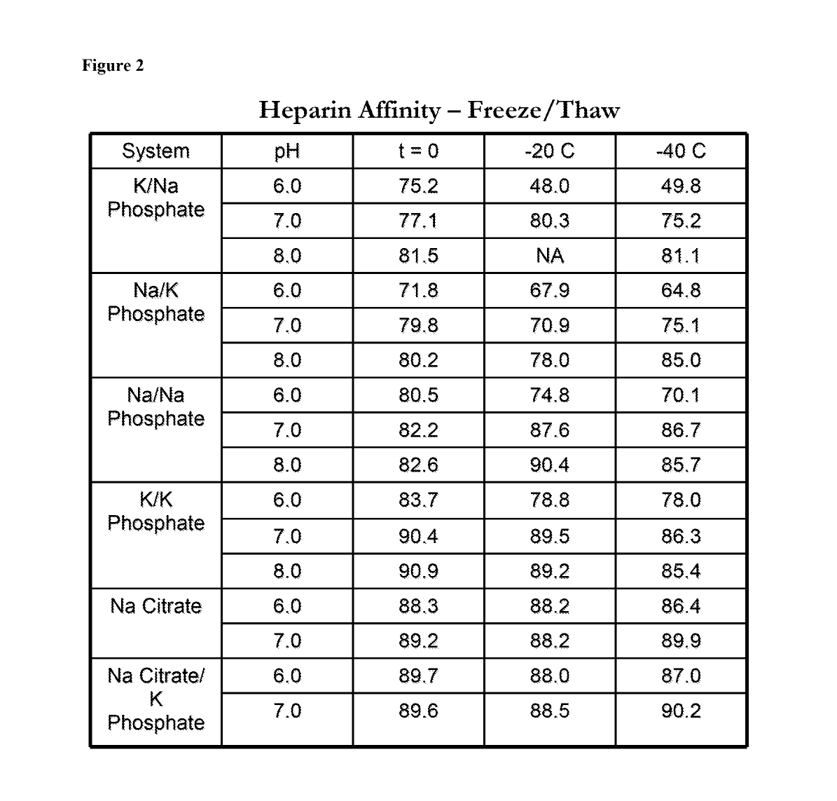

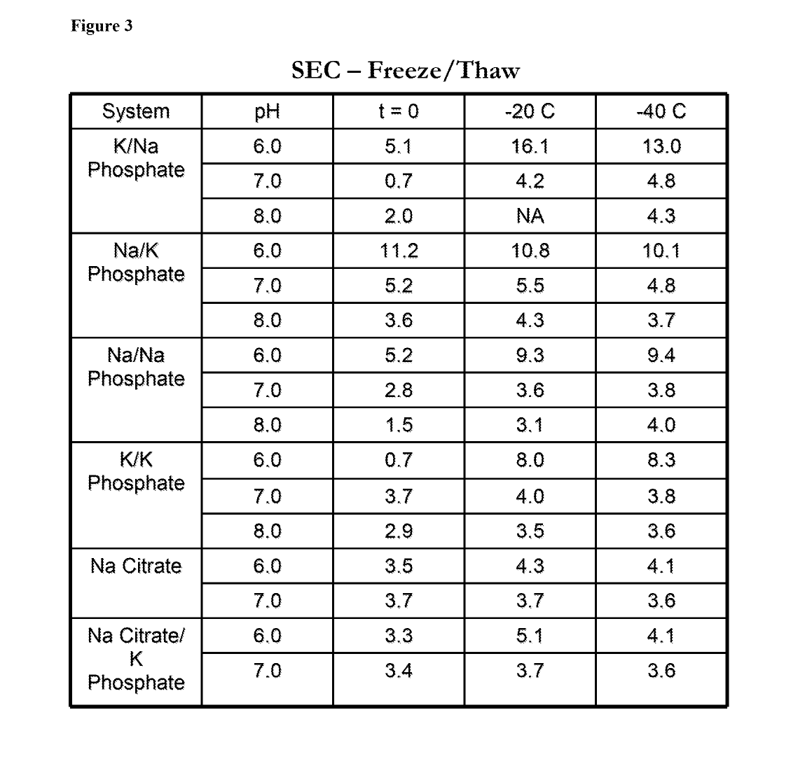

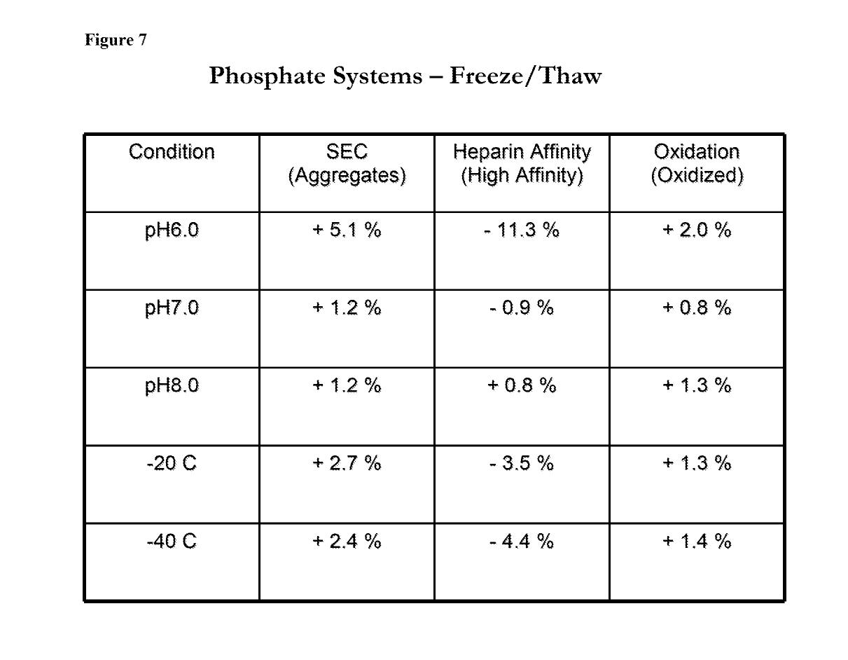

Solutions comprising antithrombin and a variety of phosphate and citrate buffers at pH 6, pH 7, or pH 8 (phosphate buffers) or at pH 6 or pH 7 (citrate buffers), were subjected to a freeze thaw cycle to −20° C. or −40° C. The solutions were kept in 60 ml bags during the freeze-thaw cycle. The concentration of antithrombin used is between 5-10 mg/ml. The oxidation status, heparin affinity, and aggregation of antithrombin were determined prior to and after undergoing the freeze-thaw cycle. The aggregation of antithrombin (expressed in percentages) was determined by Size Exclusion Chromatography (SEC). The oxidation of antithrombin was determined by using RP-HPLC to isolate the antithrombin followed by peptide mapping. FIG. 1 shows the oxidation status of antithrombin after freeze/thaw in a variety of buffers. FIG. 2 shows the heparin affinity of antithrombin after freeze/thaw in a variety of buffers. FIG. 3 shows the aggregation of antithrombin after freeze/thaw in a variety of buffers. FIG. 7 provides an overview of the stability parameters of antithrombin in phosphate systems after freeze/thaw.

### Example 2

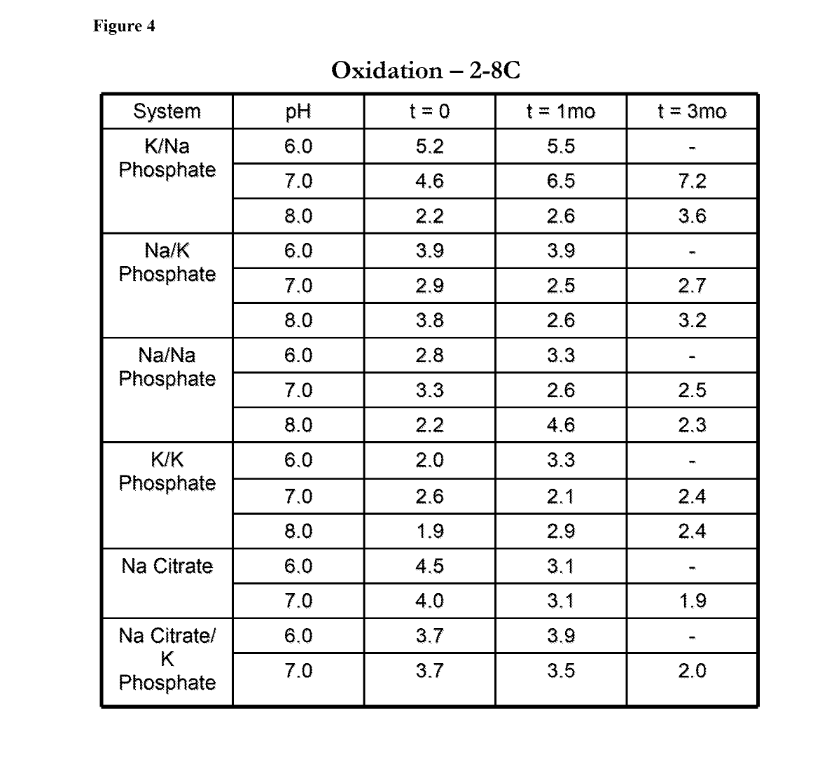

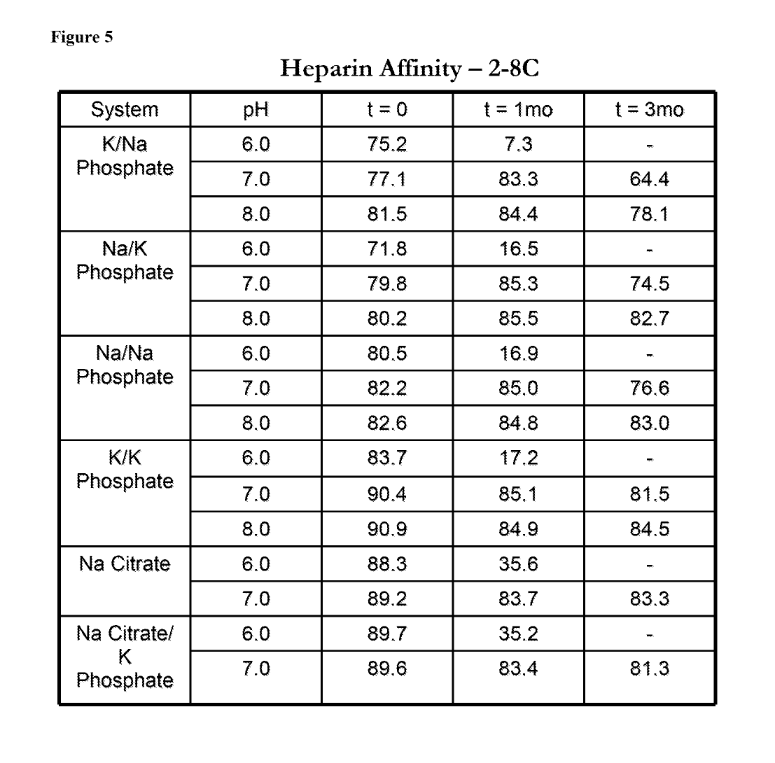

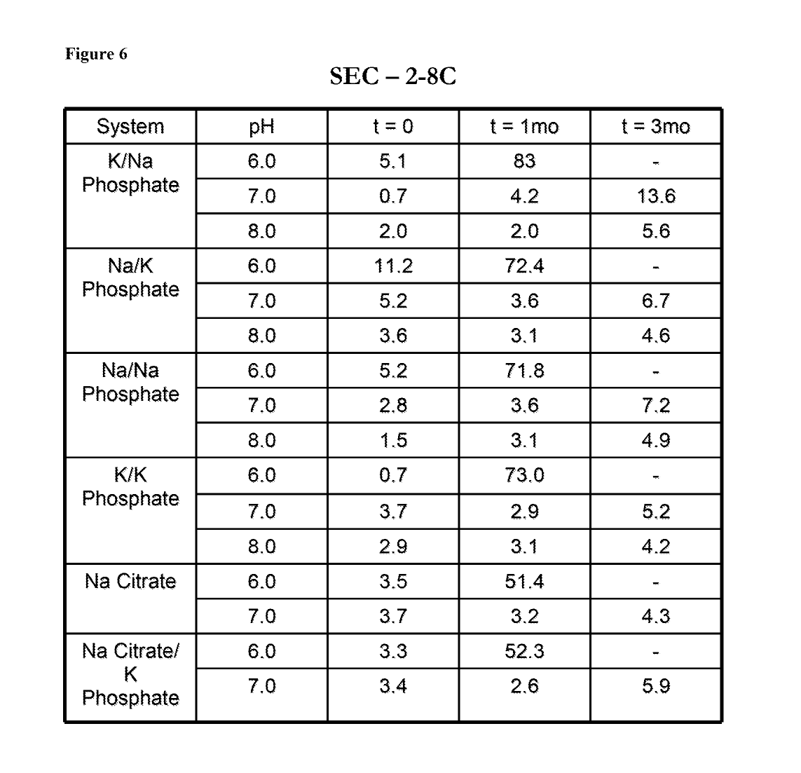

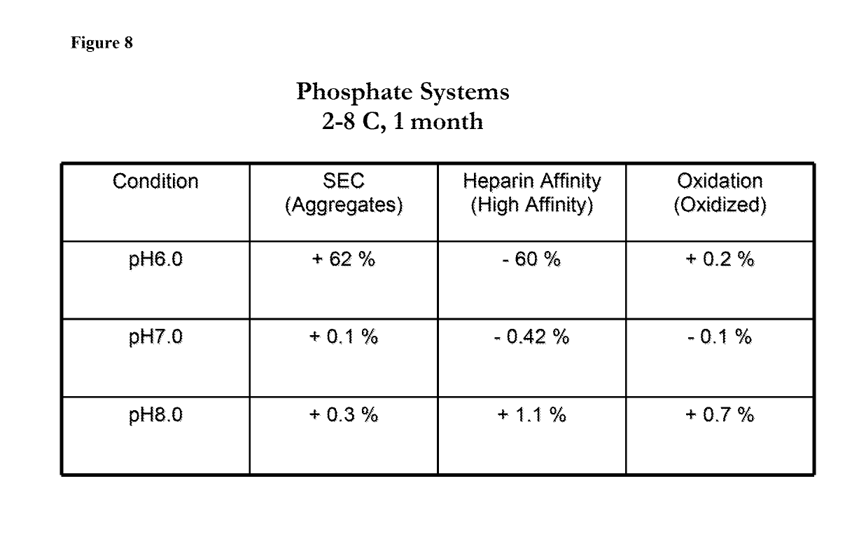

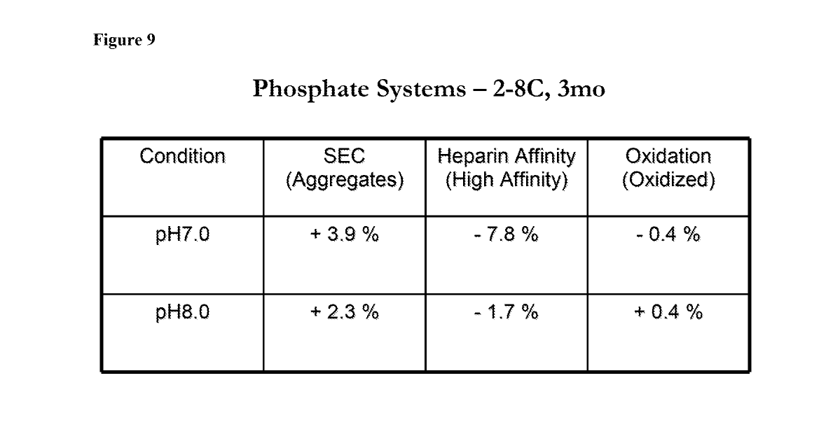

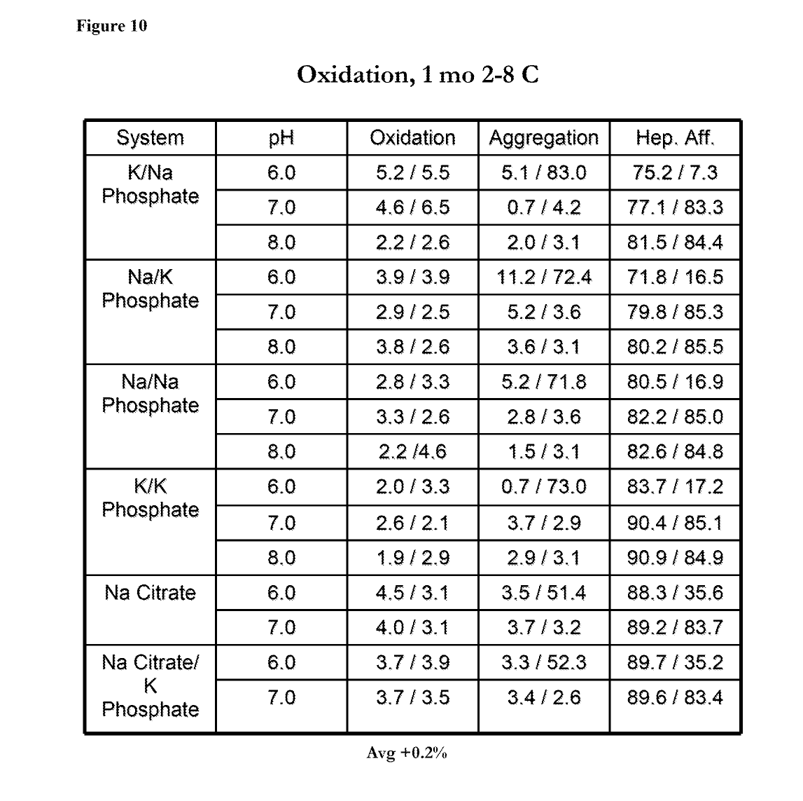

Solutions comprising antithrombin and a variety of phosphate and citrate buffers at pH 6, pH 7, or pH 8 (phosphate buffers) or at pH 6 or pH 7 (citrate buffers), were stored at between 2° C. and 8° C. for a period of up to three months. The solutions were stored in 60 ml bags. The concentration of antithrombin used is between 5-10 mg/ml. The oxidation status, heparin affinity and aggregation (by SEC) of antithrombin were determined prior to and after storage. The oxidation of antithrombin (expressed in percentages) was determined by using RP-HPLC to isolate the antithrombin followed by peptide mapping. The heparin binding was determined by contacting the formulation with a heparin binding column followed by HPLC. FIG. 4 shows the oxidation status of antithrombin after storage at 2-8° C. in a variety of buffers. FIG. 5 shows the heparin affinity of antithrombin after storage at 2-8° C. in a variety of buffers. FIG. 6 shows the aggregation of antithrombin after storage at 2-8° C. in a variety of buffers. FIG. 8 provides an overview of the stability parameters of antithrombin after storage at 2-8° C. for one month in phosphate systems. FIG. 9 provides an overview of the stability parameters of antithrombin after storage at 2-8° C. for three months in phosphate systems. FIG. 10 provides an overview of the stability parameters of antithrombin after storage at 2-8° C. for one month in the various buffers.

### Example 3

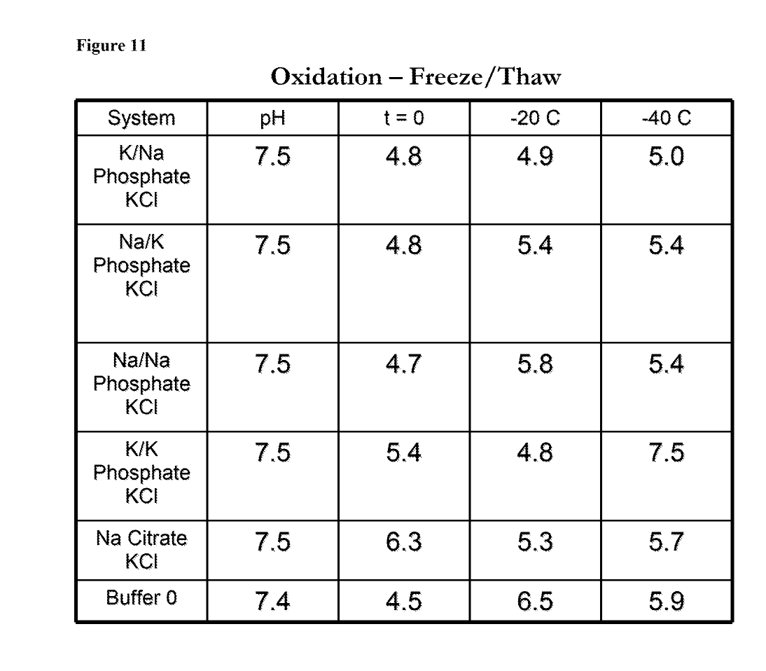

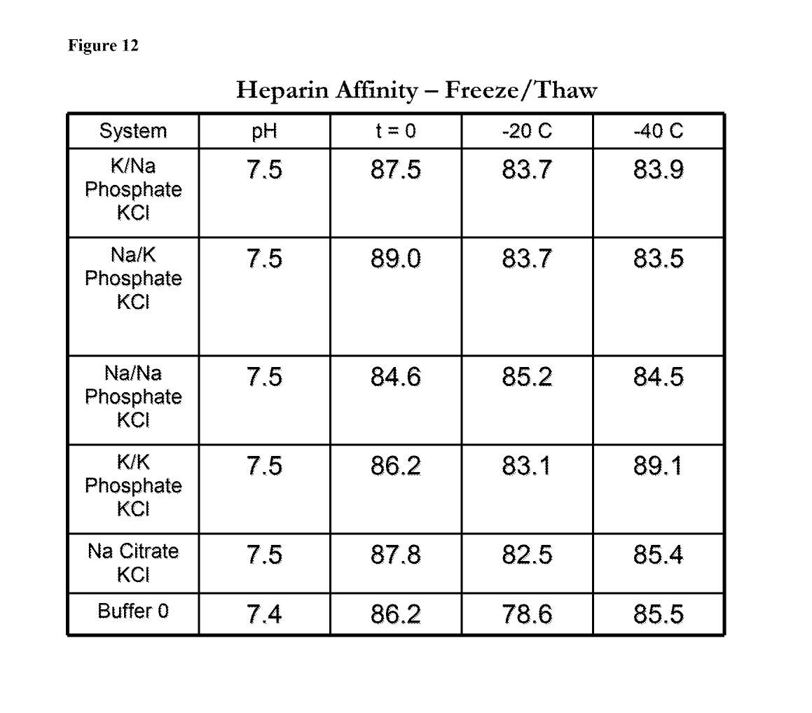

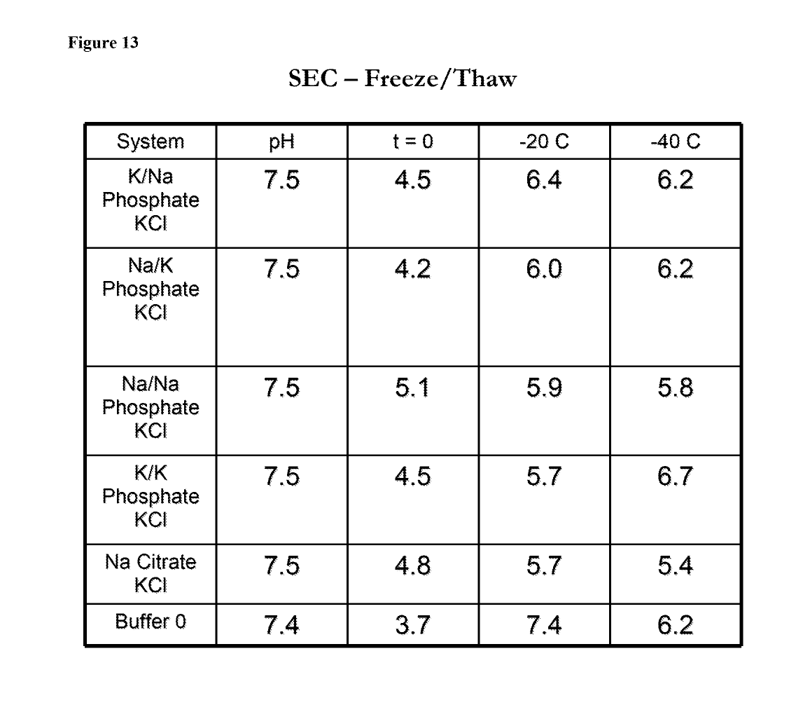

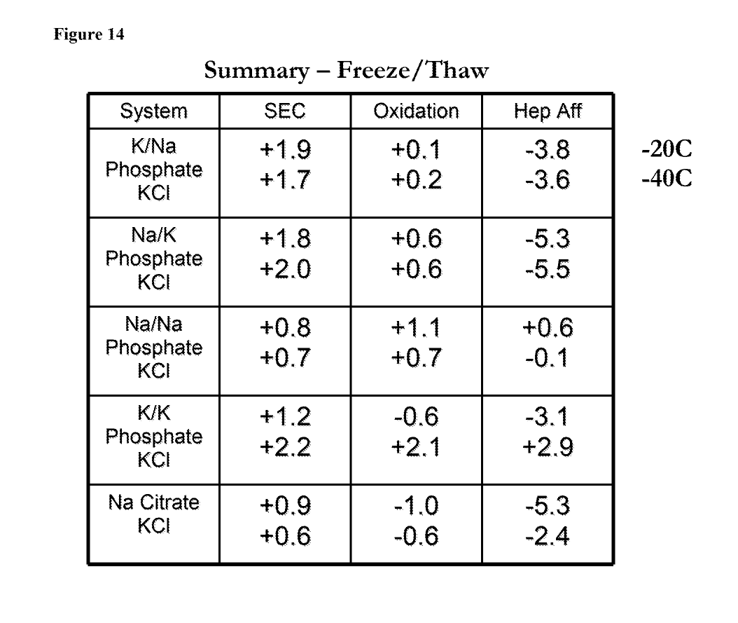

Potassium chloride (120 mM at pH 7.5) was added to solutions comprising antithrombin and a variety of phosphate and citrate buffers at pH 6, pH 7, or pH 8 (phosphate buffers) or at pH 6 or pH 7 (citrate buffers). The solutions were subsequently subjected to a freeze thaw cycle to −20° C. or −40° C. The solutions were kept in 60 ml bags during the freeze-thaw cycle. The concentration of antithrombin used is between 5-10 mg/ml. The oxidation status, heparin affinity, and aggregation of antithrombin were determined prior to and after undergoing the freeze-thaw cycle. The oxidation of antithrombin (expressed in percentages) was determined by using RP-HPLC to isolate the antithrombin followed by peptide mapping. The heparin binding was determined by contacting the formulation with a heparin binding column followed by HPLC. The aggregation of antithrombin (expressed in percentages) was determined by Size Exclusion Chromatography (SEC). FIG. 11 shows the oxidation status of antithrombin after freeze/thaw in a variety of buffers. FIG. 12 shows the heparin affinity of antithrombin after freeze/thaw in a variety of buffers. FIG. 13 shows the aggregation of antithrombin after freeze/thaw in a variety of buffers. FIG. 14 provides an overview of the stability parameters of antithrombin in the various buffers after freeze/thaw.

### Example 4

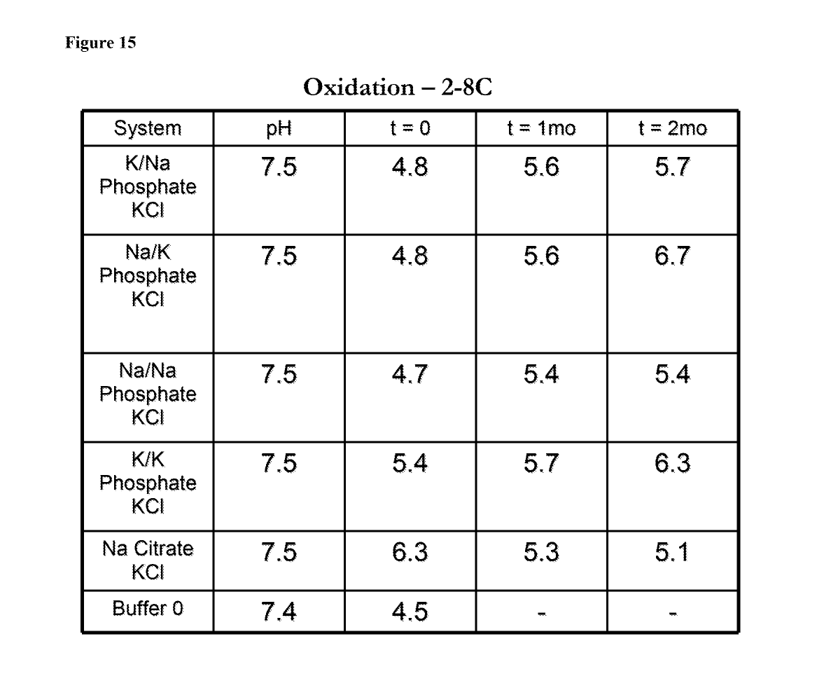

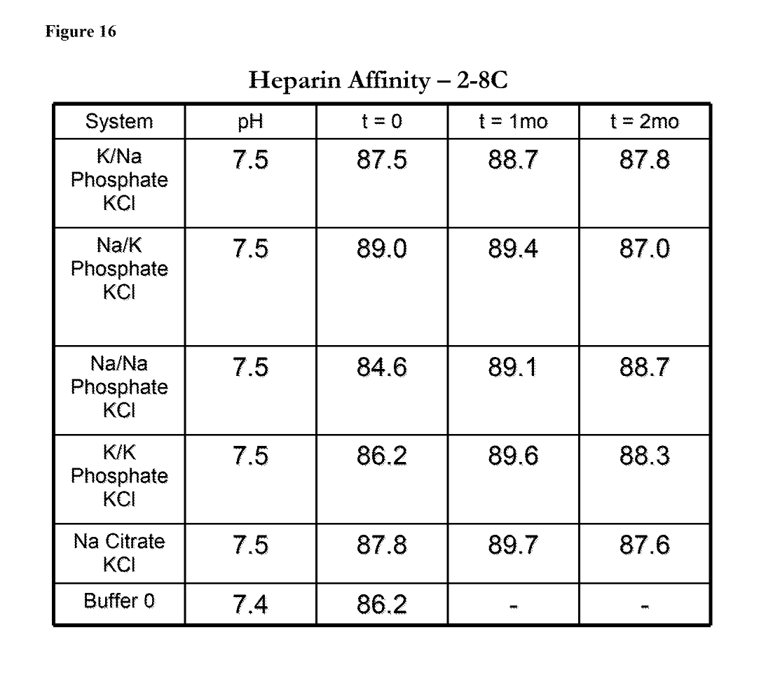

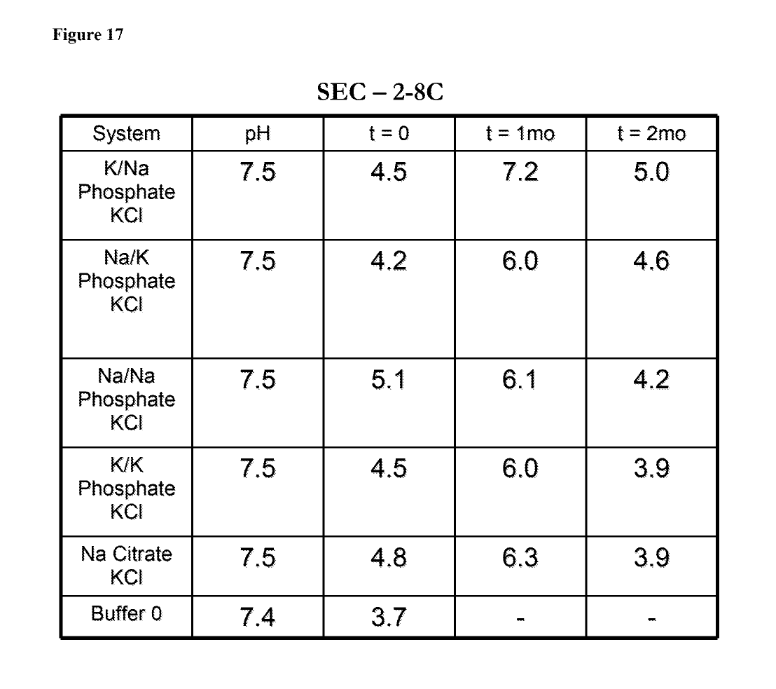

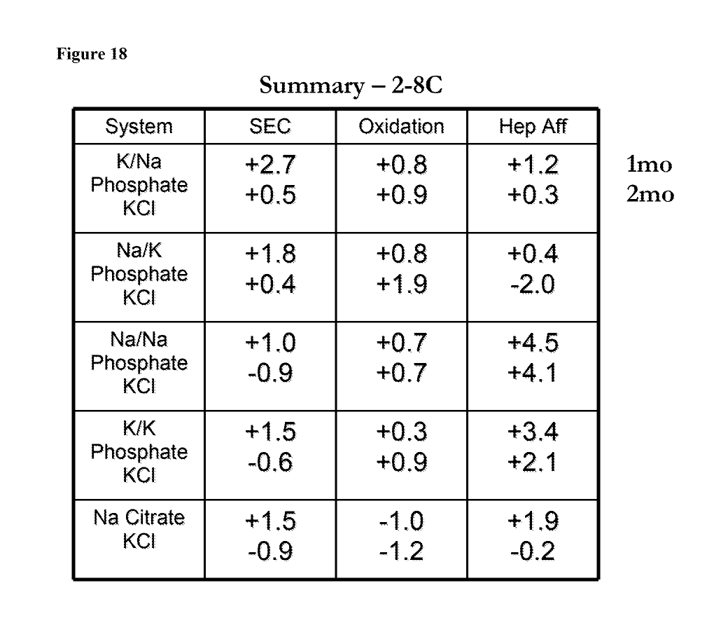

Potassium chloride (120 mM at pH 7.5) was added to solutions comprising antithrombin and a variety of phosphate and citrate buffers at pH 6, pH 7, or pH 8 (phosphate buffers) or at pH 6 or pH 7 (citrate buffers). The solutions were stored at between 2° C. and 8° C. for a period of up to three months. The solutions were stored in 60 ml bags. The oxidation status, heparin affinity and aggregation (by SEC) of antithrombin were determined prior to and after storage. The oxidation of antithrombin (expressed in percentages) was determined by using RP-HPLC to isolate the antithrombin followed by peptide mapping. The heparin binding was determined by contacting the formulation with a heparin binding column followed by HPLC. FIG. 15 shows the oxidation status of antithrombin after storage at 2-8° C. in a variety of buffers. FIG. 16 shows the heparin affinity of antithrombin after storage at 2-8° C. in a variety of buffers. FIG. 17 shows the aggregation of antithrombin after storage at 2-8° C. in a variety of buffers. FIG. 18 provides an overview of the stability parameters of antithrombin after storage at 2-8° C. in the various buffers.

### Example 5

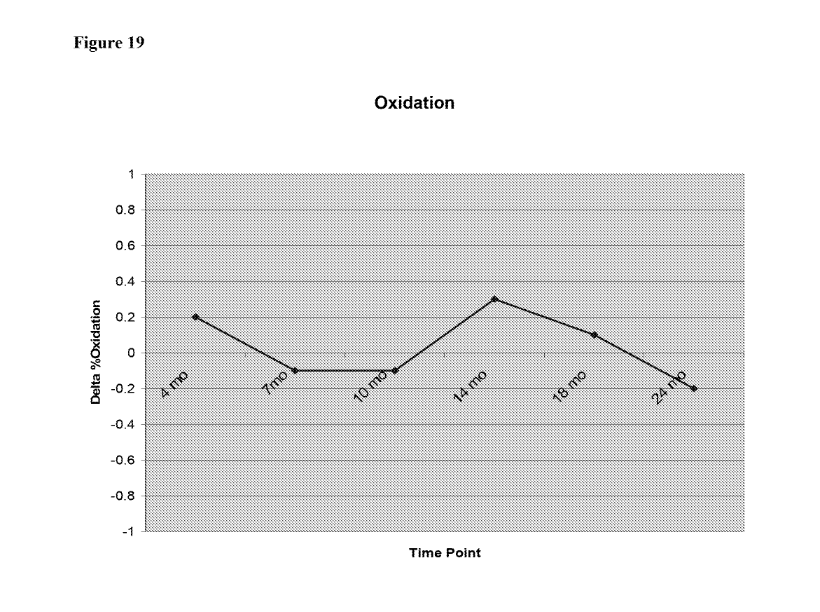

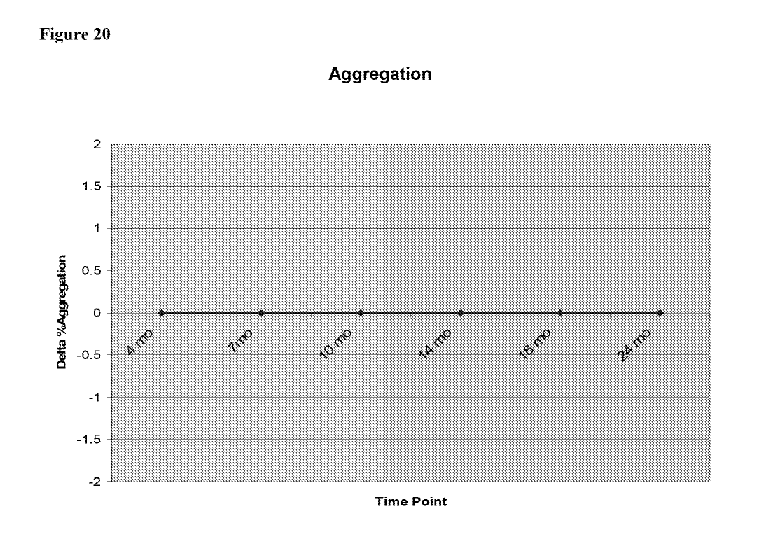

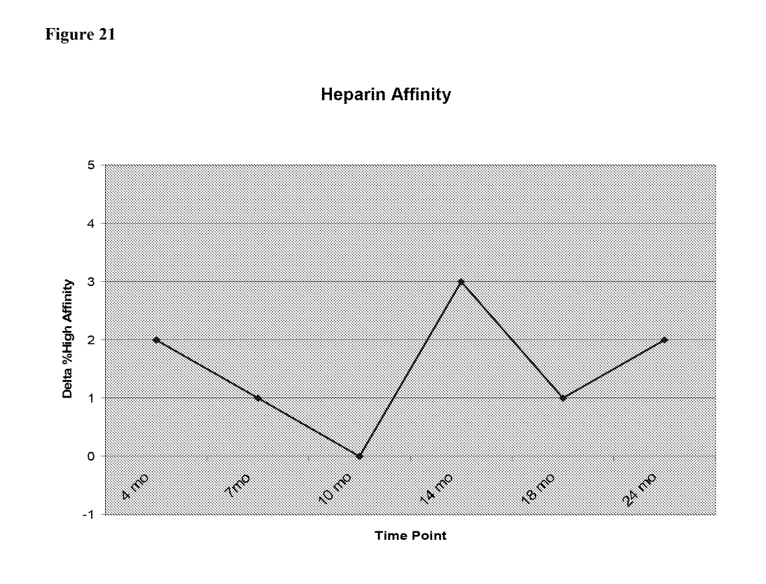

Three lots of Clarified Starting Material (CSM) were prepared at pilot scale. Each of these CSM's was frozen in 10 L bags at −20° C. and stored for up to two years. At various time points a bag was removed from the freezer, thawed and purified. The stability of the antithrombin alfa molecule was determined by monitoring oxidation, aggregation and heparin affinity over the course of the study. No significant change in any of the stability indicating parameters was observed over a two year period of frozen storage (See FIGS. 19-21).

Transgenic goat milk was clarified, pasteurized and concentrated and then sent to a storage facility until needed in a purification campaign. Initially, the CSM was formulated in PBS pH7.4 (50 mM sodium phosphate, 150 mM sodium chloride) but, upon freezing at −20° C., the solution resulted in aggregation and loss of heparin affinity upon thawing.

PBS formulations made with potassium salts at pH greater than 7 stabilized antithrombin alfa and were fully frozen at −20° C. The process was scaled up using the new clarified formulation (50 mM potassium phosphate, 120 mM potassium chloride pH8.0) to determine whether the bulk freezing impacted the product.

Three lots of CSM were produced by depth filtration, pasteurization and concentration/diafiltration into the potassium CSM formulation buffer. Prior to freezing, a small sample was removed for small scale purification to determine the time zero analytical results. Each lot was split into two approximately 5 L segments and filtered into 10 L bags. The bags were immediately frozen at −20° C. and stored until the appropriate time point was reached. A summary of the testing schedule was recorded in Table 1

TABLE 1
CSM Frozen Stability Schedule

| Time Point | CSM Lot |
|---|---|
| 4 Months | 033007 |
| 7 Months | 040507 |
| 10 Months | 033007 |
| 14 Months | 040507 |
| 18 Months | *041307 |
| 24 Months | 041307 |

*Lot was thawed for sampling and refrozen at the 7 month time point

Each time point an aliquot was thawed in a water bath and purified as described below. The CSM was loaded onto a 1.15 L Heparin HyperD and two loading/eluting cycles were performed. The two elution peaks were collected together and the pool was concentrated and diafiltered into Q Sepharose loading buffer. The product was loaded onto a 490 ml Q Sepharose column and eluted with an increased salt buffer. The Q elution was mixed 1:1 with 1.76 M sodium citrate pH7.0 and loaded directly onto a 450 ml Tosoh Phenyl 650C column. The product, which was in the flow through fraction, was collected and concentrated to approximately 25 g/l and diafiltered into Drug Substance (DS) formulation buffer. The final DS was aseptically filtered prior to analytical testing.

The final DS was used for determination of aggregation and heparin affinity while the oxidation level was determined on the heparin eluate since the oxidation increases significantly after the phenyl column. The introduction of the SP Sepharose precolumn eliminates any downstream oxidation so each time point was purified equivalently without the SP Sepharose column. All analytical results were compared to the time zero results obtained for each CSM lot.

### Materials and Methods

Each purification was performed as described in the second generation development report1 with the exception of the SP Sepharose precolumn.

Heparin HyperD (used by Cambrex 2003-2004)

Q Sepharose FF lot 303367

Tosoh Phenyl 650C lot 65PHC01B

The aggregation, heparin affinity and oxidation were run in PAD.

50 mM phosphate (K/Na) 120 mM KCl pH 7.5

50 mM phosphate (Na/K) 120 mM KCl pH 7.5

50 mM phosphate (Na/Na) 120 mM KCl pH 7.5

50 mM phosphate (K/K) 120 mM KCl pH 7.5

50 mM sodium citrate 120 mM KCl pH 7.5

### Results

Each frozen bag was carefully inspected for signs of incomplete freezing and/or pooling prior to thawing. There were no observed anomalies at any time point. Table 2 contains a summary of the analytical results throughout the stability study.

TABLE 2
CSM Frozen Stability Analytical Results

| | Oxidation of | | | | | |
|---|---|---|---|---|---|---|
| | Heparin Eluate | | Aggregation | | Heparin Affinity | |
| Time Point | t = 0 | t = X | t = 0 | t = X | t = 0 | t = X |
| 4 Months | 5.7% | 5.9% | <0.1% | <0.1% | 97% | 99% |
| 7 Months | 5.7% | 5.6% | <0.1% | <0.1% | 97% | 98% |
| 10 Months | 5.7% | 5.6% | <0.1% | <0.1% | 97% | 97% |
| 14 Months | 5.7% | 6.0% | <0.1% | <0.1% | 97% | 100% |
| 18 Months | 3.8% | 3.9% | <0.1% | <0.1% | 96% | 97% |
| 24 Months | 3.8% | 3.6% | <0.1% | <0.1% | 96% | 98% |

Since each lot had different time zero results, the data was normalized to display the difference in each analytical result relative its respective time zero result. The data for each stability-indicating technique was plotted in FIGS. 19-21.

The oxidation results fluctuated from an increase of 0.3% to a decrease of 0.2%. These fluctuations average out and the net difference was negligible relative to the initial time point. Each of the heparin affinity results were equal to or higher than the time zero result. Therefore, frozen storage at −20° C. does not adversely affect antithrombin alfa. The aggregation results never exceeded the limit of quantitation of the assay for each of the time points as well as the initial, unfrozen sample. Storage at −20° C. had no effect on the aggregation of antithrombin alfa.

### Conclusion

The potassium phosphate buffered potassium chloride clarified formulation buffer froze completely at −20° C. as the temperature is sufficiently lower than the lowest eutectic point of the solution. The sodium based PBS used previously remained partially liquid due to the sodium chloride which caused extremely high levels of aggregation and low levels of heparin affinity over time. Replacement of the sodium with potassium eliminated this problem.

The stability of antithrombin alfa in the frozen state at −20° C. was demonstrated for up to two years in the all potassium buffer. The quality of the product was assessed by the three most sensitive stability-indicating assays. Each stability time point preparation was comparable, by all three assays, to the initial small scale purification performed on the fresh CSM. Therefore, Clarified Starting Material was determined to be stable in 50 mM potassium phosphate, 120 mM potassium chloride pH8.0 frozen at −20° C. for up to 24 months.

### Example 6

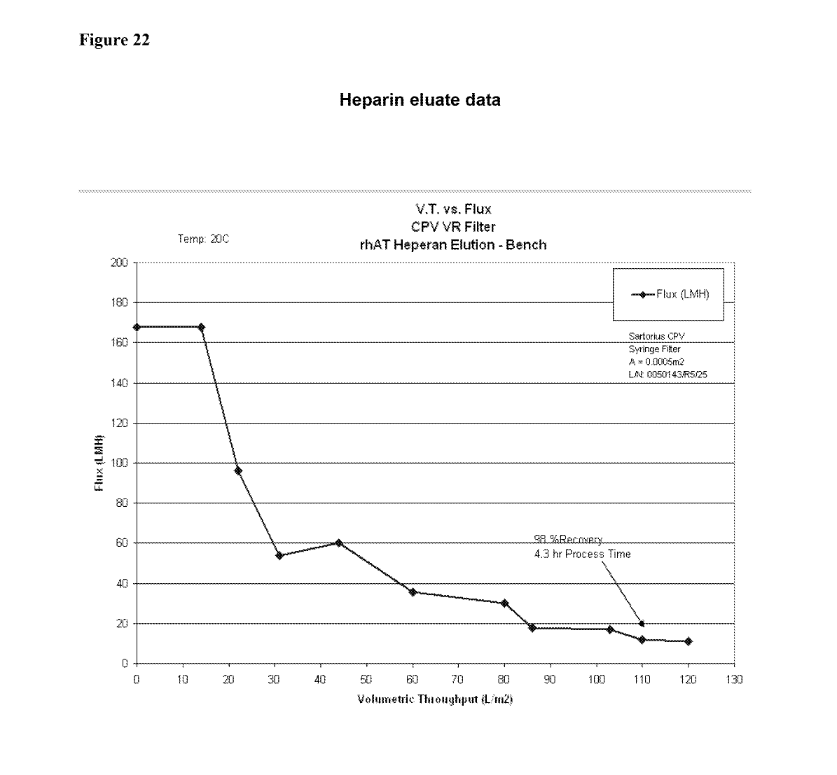

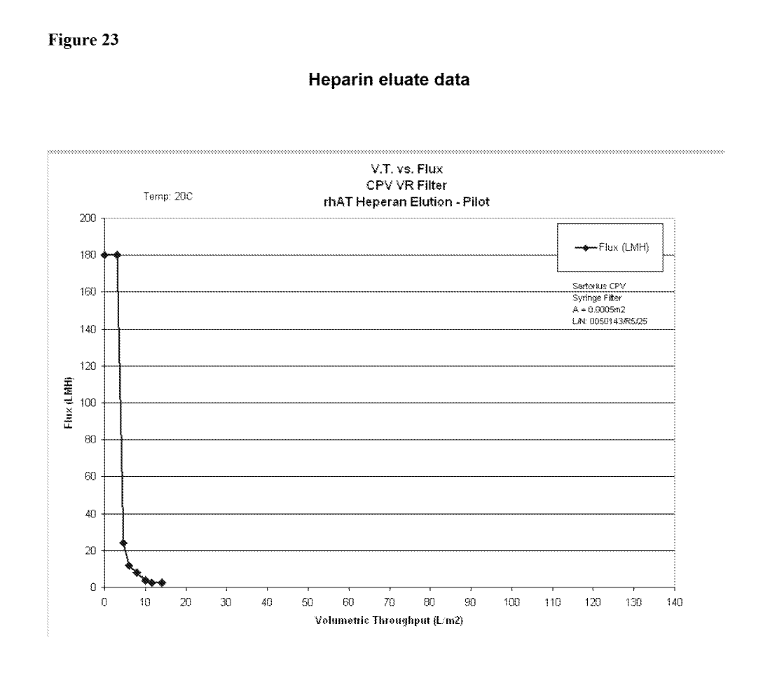

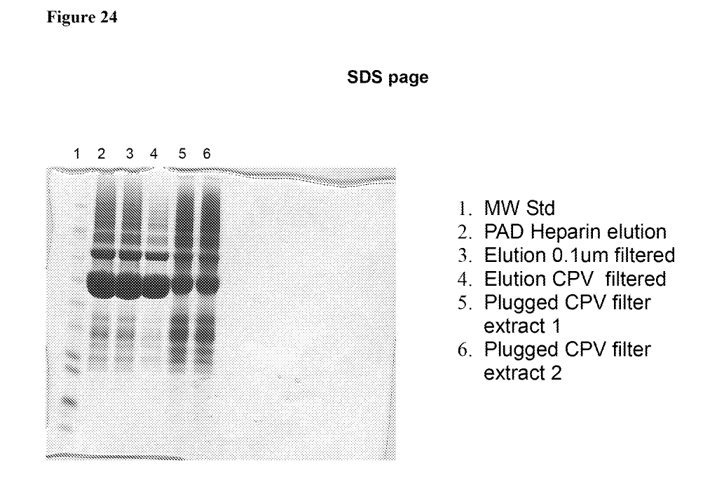

Nanofiltration was performed for removal of virus from the antithrombin formulation. The clarified milk pool was purified using a heparin column. The heparin eluate was filtered using 5 cm² 20 nM viral filters. The streams were analyzed using SDS Page. Prefilters were tested to remove fouling species: 0.1 um PES Pre-filter, 0.2 uM Depth filter, 300 KD UF, Q-absorber and S-absorber. The throughput data are shown in FIGS. 22 and 23. The SDS page is shown in FIG. 24.

### EQUIVALENTS

The foregoing written specification is considered to be sufficient to enable one skilled in the art to practice the invention. The present invention is not to be limited in scope by examples provided, since the examples are intended as an illustration of certain aspects and embodiments of the invention. Other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

What is claimed is:

1. A formulation comprising a therapeutic protein and a buffer,
wherein the buffer comprises potassium mono-hydrogen-phosphate and potassium di-hydrogen-phosphate, or
wherein the buffer comprises sodium mono-hydrogen-phosphate and sodium di-hydrogen-phosphate.

2. The formulation of claim 1, wherein the buffer has a concentration of between 10 mM and 100 mM.

3. The formulation of claim 2, wherein the buffer has a concentration of 50 mM.

4. The formulation of claim 1, further comprising potassium chloride.

5. The formulation of claim 4, wherein the potassium chloride has a concentration of between 100 and 150 mM.

6. The formulation of claim 4, wherein the potassium chloride has a concentration of 120 mM.

7. The formulation of claim 1, wherein the pH of the formulation is between 7.5 and 8.5.

8. The formulation of claim 7, wherein the pH of the formulation is 8.

9. The formulation of claim 1, wherein the therapeutic protein is antithrombin.

10. The formulation of claim 1, wherein the formulation comprises a clarified milk product.

11. The formulation of claim 1, wherein the formulation includes additional proteins.

12. The formulation of claim 9, wherein the antithrombin maintains at least 90% of heparin binding functionality after storage at 2-8° C. for three months as compared to heparin binding functionality prior to storage.

13. The formulation of claim 12, wherein the increase in the amount of antithrombin (by weight) that is in an aggregated form after storage at 2-8° C. for three months is less than 3-fold as compared to the amount of antithrombin (by weight) that is in an aggregated form prior to storage.

14. The formulation of claim 12, wherein the increase in the amount of oxidation of antithrombin after storage at 2-8° C. for three months is less than 2-fold compared to the amount of oxidation of antithrombin prior to storage.

15. A method for generating a formulation that stabilizes a therapeutic protein, the method comprising:
providing a solution comprising a buffer,
wherein the buffer comprises potassium mono-hydrogen-phosphate and potassium di-hydrogen-phosphate, or
wherein the buffer comprises sodium mono-hydrogen-phosphate and sodium di-hydrogen-phosphate, and
adding the therapeutic protein to the solution resulting in a formulation that stabilizes the therapeutic protein.

16. A method for generating a formulation that stabilizes a therapeutic protein, the method comprising:
providing a solution comprising the therapeutic protein, and adding a buffer to the solution resulting in a formulation that stabilizes the therapeutic protein,
wherein the buffer comprises potassium mono-hydrogen-phosphate and potassium di-hydrogen-phosphate, or
wherein the buffer comprises sodium mono-hydrogen-phosphate and sodium di-hydrogen-phosphate.

17. The method of claim 15, wherein the resulting concentration of the buffer is 50 mM.

18. The method of claim 15, wherein the formulation further comprises potassium chloride.

19. The method of claim 15, wherein the resulting pH of the solution is a pH of 8.

20. The method of claim 15, wherein the therapeutic protein is antithrombin.

21. A method for generating a formulation that stabilizes antithrombin, the method comprising:
separating antithrombin from a milk composition comprising antithrombin resulting in a solution comprising antithrombin,
pasteurizing the solution comprising antithrombin,
exchanging the solution comprising antithrombin for a buffer,
wherein the buffer comprises potassium mono-hydrogen-phosphate and potassium di-hydrogen-phosphate, or
wherein the buffer comprises sodium mono-hydrogen-phosphate and sodium di-hydrogen-phosphate,
thereby generating a formulation that stabilizes antithrombin.
